SEQUENCE LISTING (2 sequences)

SEQ ID NO: 1 - protein, 11 aa, Artificial Sequence
  His-tag for PLK kinase domain
MKKGHHHHHHD

SEQ ID NO: 2 - protein, 9 aa, Artificial Sequence
  Optimized PLK Peptide Substrate
SFNDTLDFD

(19) United States
(12) Patent Application Publication
(10) Pub. No.: US 2009/0005398 A1
(43) Pub. Date: Jan. 1, 2009

(54) Methods For The Treatment of Central Nervous System Tumors

(76) Inventor: Mohammed Dar, Durham, NC (US)

Correspondence Address: GLAXOSMITHKLINE;Corporate Intellectual Property, Five Moore Drive, P.O. Box 13398, Research Triangle Park, NC, 27709-3398, US

(21) Appl. No.: 12/142,845

(22) Filed: Jun. 20, 2008

Related U.S. Application Data
(60) Provisional application No. 60/946,513, filed on Jun. 27, 2007.
(60) Provisional application No. 60/949,946, filed on Jul. 16, 2007.

(51) Int. Cl. A61K 31/497; A61P 35/00

(52) U.S. Cl. 514/254.06

(57) ABSTRACT

Methods for the treatment of primary and secondary central nervous system tumors in a mammal which comprise administration of a benzimidazole thiophene compound are provided.

### CROSS-REFERENCE TO RELATED APPLICATIONS

This application claims priority to U.S. Provisional Patent Application Ser. Nos. 60/949,946 filed 16 Jul. 2007 and 60/946,513 filed 27 Jun. 2007.

### BACKGROUND OF THE INVENTION

The present invention relates to novel methods for the treatment of primary central nervous system (CNS) tumors and secondary CNS tumors (i.e., metastases in the CNS, from a tumor originating outside of the CNS) with benzimidazole thiophene compounds. Tumors of the CNS may be categorized as primary CNS tumors or secondary CNS tumors. Primary CNS tumors are neoplasms that originate in the brain, spinal cord, or the lining around such structures (e.g., meninges and Schwan cells). Primary CNS tumors include tumors of neuroepithelial tissue such as astrocytic tumors, oligodendroglial tumors, mixed gliomas, ependymal tumors, choroid plexus tumors, neuronal and mixed neuronal-glial tumors, neuroblastic tumors, pineal parenchymal tumors, and embryonal tumors; tumors of peripheral nerves such as schwannoma (neurinoma), neurofibroma, perineurioma and malignant peripheral nerve sheath tumor (MPNST); tumors of the meninges (also known as “meningeal tumors”) such as tumors of meningothelial cells, mesenchymal non-meningothelial tumors and primary melanocytic lesions; lymphomas and hemopoietic neoplasms such as malignant lymphomas, plasmacytoma and granulocytic sarcoma; germ cell tumors such as germinoma, embryonal carcinoma, yolk sac tumor, choriocarcinoma, teratoma and mixed germ cell tumors; and tumors of the sellar region such as craniopharyngioma and granular cell tumor.

The term glioma refers to tumors believed to be derived from normal glial cells (i.e., astrocytes, oligodendrocytes, and ependymal cells). For each of these cell types, there is a malignant counterpart (e.g., astrocytoma is derived from astrocytes). Astrocytic tumors comprise over 80% of primary CNS tumors and are classified by the type of cell they most closely resemble and according to their clinical and biological behavior (i.e., tumor grade). The slower growing lesions are commonly referred to as “low-grade gliomas” (LGGs), while the more clinically aggressive tumors are classified as “high-grade gliomas” (HGGs). HGGs are more common comprising nearly 80% of all gliomas.

Astrocytic tumors, the most common type of neuroepithelial tissue tumors (and are therefore sometimes loosely referred to by the term “glioma”), can be further subdivided based on the severity of the condition (i.e., WHO Grade 1 to 4, based on the severity of the condition, with 4 being the most serious form of glioma). Grade 1 corresponds to pilocytic astrocytoma; Grade 2 corresponds to diffuse astrocytoma; Grade 3 corresponds to anaplastic or malignant astrocytoma; and Grade 4 corresponds to glioblastoma multiforme, which is the most common glioma in adults and is considered the most serious form of astrocytic tumor.

Secondary CNS tumors are the most common form of brain tumors. These tumors originate outside of the CNS and result from the primary tumor metastasizing to the CNS. Secondary CNS tumors can either involve the brain directly (i.e., parenchymal involvement) or involve the lining (i.e., leptomeningeal and meningeal involvement). The meningeal lining is composed of three adjacent tissue layers (in order of farthest to nearest to the brain surface): dura mater, arachnoid membrane, and pia mater. Carcinomatous meningitis is a condition in which tumor cells spread and involve the leptomeningeal space (i.e., space between the pia mater and arachnoid membrane). In adults, solid tumors that have been shown to frequently metastasize to the CNS include lung, breast, adenocarcinoma of unknown primary site, melanoma, renal, and colon cancer. In children, primary solid tumors that more commonly metastasize to the CNS include sarcoma, Wilm's tumor, neuroblastoma, and germ cell tumor. In addition to solid tumors, haematological malignancies that can metastasize to the CNS include acute lymphoblastic leukemia, high grade non-Hodgkin's lymphoma, and less commonly acute myeloid leukemia.

Treatment of primary and secondary CNS tumors depends on the multiplicity, location, and grade of the tumor. Treatment of secondary CNS tumors may also depend upon the status of the systemic tumor. Treatment may include any of surgical resection, stereotactic radiosurgery (SRS), whole brain radiotherapy (WBRT) and chemotherapy or some combination thereof. Currently there is no consensus on optimal treatment in instances where CNS progression after WBRT and/or SRS occurs; possible options are to attempt or re-attempt SRS or to utilize chemotherapy.

The inability of many conventional chemotherapeutic agents to cross the blood-brain barrier (BBB) has historically limited their use in the treatment of CNS tumors. The BBB is formed by the complex tight junctions between the endothelial cells of the brain capillaries and their low endocytic activity (Potschka et al., Journal of Pharm. and Exp. Therapeutics 306(1):124-131, 2003 July). This results in a capillary wall that behaves as a continuous lipid bilayer and prevents the passage of polar and lipid-insoluble substances. Additionally, ATP-dependent multidrug transporters such as P-glycoprotein (Pgp; ABCB1) and multidrug resistance protein MRP2 (ABCC2), which are found in the membranes of brain capillary endothelial cells, are thought to play an important role in BBB function by limiting drug penetration into the brain. It is, therefore, an important obstacle to drugs that may combat diseases affecting the CNS.

Brain tumors may disrupt the function of the BBB locally and nonhomogeneously. The effect of such disruption may explain CNS activity observed with a variety of chemotherapeutic regimens (cytoxan/methotrexate/5-fluorouracil, doxorubicin/cytoxan, capecitabine, cisplatin/etoposide), despite the fact that none of these agents cross the intact BBB at the doses used. In two autopsy studies, clinically relevant concentrations of platins were present within brain tumors, but not adjacent normal brain, supporting the hypothesis that the blood-tumor barrier has very distinct properties than the intact blood brain barrier (Stewart D. J. et al., Am. J. Clin. Oncol. 11:152-158, 1988; and Stewart D. J. et al., Cancer Res. 42:2472-2479).

Because of the difficulty in treating primary and secondary CNS tumors and the limitations of existing treatment options, there means a need in the art for chemotherapeutic agents for the treatment of such tumors.

Polo-like kinases (“PLK”) are evolutionarily conserved serine/threonine kinases that play critical roles in regulating processes in the cell cycle. PLK plays a role in the entry into and the exit from mitosis in diverse organisms from yeast to mammalian cells. PLK includes PLK1, PLK2, PLK3 and PLK4. Overexpression of PLK1 appears to be strongly associated with neoplastic cells (e.g., cancers).

PCT Publication No. WO2004/014899 to SmithKline Beecham discloses novel benzimidazole thiophene compounds of formula (I):

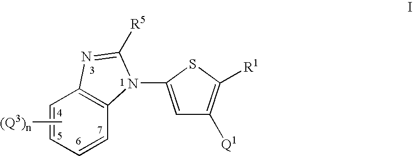

wherein:
R¹ is selected from the group consisting of H, alkyl, alkenyl, alkynyl, —C(O)R⁷, —CO₂R⁷, —C(O)NR⁷R⁸, —C(O)N(R⁷)OR⁸, —C(O)N(R⁷)—R²—OR⁸, —C(O)N(R⁷)-Ph, —C(O)N(R⁷) —R²-Ph, —C(O)N(R⁷)C(O)R⁸, —C(O)N(R⁷)CO₂R⁸, —C(O)N(R⁷)C(O)NR⁷R⁸, —C(O)N(R⁷)S(O)₂R⁸, —R²—OR⁷, —R²—O—C(O)R⁷, —C(S)R⁷, —C(S)NR⁷R⁸, —C(S)N(R⁷)-Ph, —C(S)N(R⁷)—R²-Ph, —R²—SR⁷, —C(═NR⁷)NR⁷R⁸, —C(═NR⁷)N(R⁸)-Ph, —C(═NR⁷)N(R⁸)—R²-Ph, —R²—NR⁷R⁸, —CN, —OR⁷, —S(O)_{f}R⁷, —S(O)₂NR⁷R⁸. —S(O)₂N(R⁷)-Ph, —S(O)₂N(R⁷)—R²-Ph, —NR⁷R⁸, N(R⁷)-Ph, —N(R⁷)—R²-Ph, —N(R⁷)—SO₂R⁸ and Het;
Ph is phenyl optionally substituted from 1 to 3 times with a substituent selected from the group consisting of halo, alkyl, —OH, —R²—OH, —O-alkyl, —R²—O-alkyl, —NH₂, —N(H)alkyl, —N(alkyl)₂, —CN and —N₃;
Het is a 5-7 membered heterocycle having 1, 2, 3 or 4 heteroatoms selected from N, O and S, or a 5-6 membered heteroaryl having 1, 2, 3 or 4 heteroatoms selected from N, O and S, each optionally substituted from 1 to 2 times with a substituent selected from the group consisting of halo, alkyl, oxo, —OH, —R²—OH, —O-alkyl, —R²—O-alkyl, —NH₂, —N(H)alkyl, —N(alkyl)₂, —CN and —N₃;
Q¹ is a group of formula: —(R²)ₐ—(Y¹)_{b}—(R²)_{c}—R³
a, b and c are the same or different and are each independently 0 or 1 and at least one of a or b is 1;
n is 0, 1, 2, 3 or 4;
Q² is a group of formula: —(R²)ₐₐ—(Y²)_{bb}—(R²)_{cc}—R⁴
   or two adjacent Q² groups are selected from the group consisting of alkyl, alkenyl, —OR⁷, —S(O)_{f}R⁷ and —NR⁷R⁸ and together with the carbon atoms to which they are bound, they form a C₅₋₆cycloalkyl, C₅₋₆cycloalkenyl, phenyl, 5-7 membered heterocycle having 1 or 2 heteroatoms selected from N, O and S, or 5-6 membered heteroaryl having 1 or 2 heteroatoms selected from N, O and S;
aa, bb and cc are the same or different and are each independently 0 or 1;
each Y¹ and Y² is the same or different and is independently selected from the group consisting of —O—, —S(O)_{f}—, —N(R⁷)—, —C(O)—, —OC(O)—, —CO₂—, —C(O)N(R⁷)—, —C(O)N(R⁷)S(O)₂—, —OC(O)N(R⁷)—, —OS(O)₂—, —S(O)₂N(R⁷)—, —S(O)₂N(R⁷)C(O)—, —N(R⁷)S(O)₂—, —N(R⁷)C(O)—, —N(R⁷)CO₂— and —N(R⁷)C(O)N(R⁷)—;
each R² is the same or different and is independently selected from the group consisting of alkylene, alkenylene and alkynylene;
each R³ and R⁴ is the same or different and is each independently selected from the group consisting of H, halo, alkyl, alkenyl, alkynyl, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN, —N₃ and a group of formula (II):

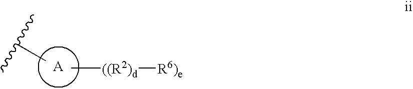

wherein:
Ring A is selected from the group consisting of C₅₋₁₀cycloalkyl, C₅₋₁₀cycyloalkenyl, aryl, 5-10 membered heterocycle having 1, 2 or 3 heteroatoms selected from N, O and 5 and 5-10 membered heteroaryl having 1, 2 or 3 heteroatoms selected from N, O and S
each d is 0 or 1;
e is 0, 1, 2, 3 or 4;
each R⁶ is the same or different and is independently selected from the group consisting of H, halo, alkyl, alkenyl, alkynyl, cycloalkyl, cycloalkenyl, Ph, Het, —CH(OH)—R²— H, —C(O)R⁷, —CO₂R⁷, —CO₂—R₂-Ph, —CO₂—R²—Het, —C(O)NR⁷R⁸, —C(O)N(R⁷)C(O)R⁷, —C(O)N(R⁷)CO₂R⁷, —C(O)N(R⁷)C(O)NR⁷R⁸, —C(O)N(R⁷)S(O)₂R⁷, —C(S)R⁷, —C(S)NR⁷R⁸, —C(═NR⁷)R⁸, —C(═NR⁷)NR⁷R⁸, —CR⁷═N—OR⁸, ═O, —OR⁷, —OC(O)R⁷, —OC(O)Ph, —OC(O)Het, —OC(O)NR⁷R⁸, —O—R²—S(O)₂R⁷, —S(O)_{f}R⁷, —S(O)₂NR⁷R⁸, —S(O)₂Ph, —S(O)₂Het, —NR⁷R⁸, —N(R⁷)C(O)R⁸, —N(R⁷)CO₂R⁸, —N(R⁷)—R²—CO₂R⁸, —N(R⁷)C(O)NR⁷R⁸, —N(R⁷)—R²—C(O)NR⁷R⁸, —N(R⁷)C(O)Ph, —N(R⁷)C(O)Het, —N(R⁷)Ph, —N(R⁷)Het, —N(R⁷)C(O)NR⁷—R²—NR⁷R⁸, —N(R⁷)C(O)N(R⁷)Ph, —N(R⁷)C(O)N(R⁷)Het, —N(R⁷)C(O)N(R⁷)—R²—Het, —N(R⁷)S(O)₂R⁸, —N(R⁷)—R²—S(O)₂R⁸, —NO₂, —CN and —N₃;
wherein when Q¹ is defined where b is 1 and c is 0, R³ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
wherein when Q² is defined where bb is 1 and cc is 0, R⁴ is not halo, —C(O)R⁷, —C(O)NR⁷R⁸, —CO₂R⁷, —C(S)R⁷, —C(S)NR⁷R⁸, —C(═NR⁷)R⁸, —C(═NR⁷)NR⁷R⁸, —CR⁷═N—OR⁷, —OR⁷, —S(O)_{f}R⁷, —S(O)₂NR⁷R⁸, —NR⁷R⁸, —N(R⁷)C(O)R⁸, —N(R⁷)S(O)₂R⁸, —NO₂, —CN or —N₃;
R⁵ is selected from the group consisting of H, halo, alkyl, cycloalkyl, OR⁷, —S(O)_{f}R⁷, —NR⁷R⁸, —NHC(O)R⁷, —NHC(O)NR⁷R⁸ and —NHS(O)₂R⁷;
f is 0, 1 or 2; and
each R⁷ and each R³ are the same or different and are each independently selected from the group consisting of H, alkyl, alkenyl, alkynyl, cycloalkyl and cycloalkenyl;
   wherein when R¹ is —CO₂CH₃ and n is 0, Q¹ is not —OH;
   or a pharmaceutically acceptable salt, solvate or physiologically functional derivative thereof.

PCT Publication No. WO2007/030361, published 15 Mar. 2007 to GlaxoSmithKline discloses, inter alia, the compound:

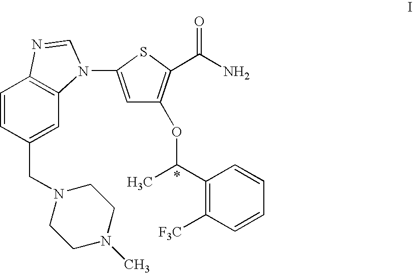

wherein * indicates a chiral carbon,
   and the enantiomerically enriched and pure form wherein the stereochemistry of the chiral carbon is R, namely the compounds of formula (I-A):

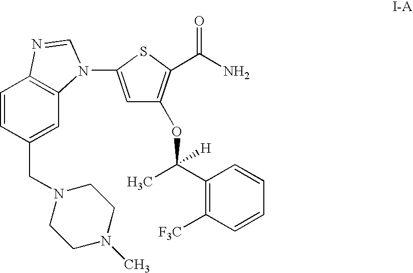

and pharmaceutically acceptable salts thereof.

Also disclosed in each of these publications are pharmaceutical compositions containing these compounds, processes for their preparation and methods for treatment of conditions mediated by PLK using these compounds, including the treatment of susceptible neoplasms.

### BRIEF SUMMARY OF THE INVENTION

According to a first aspect of the invention there is provided a method for the treatment of a primary CNS tumor in a mammal in need thereof. The method comprises administering to the mammal a therapeutically effective amount of a compound of formula (I):

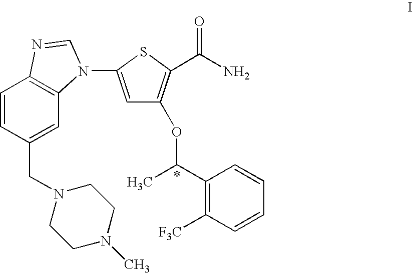

wherein * indicates a chiral carbon,
   or a pharmaceutically acceptable salt thereof.

In a second aspect, the present invention provides a method for the treatment of a tumor of neuroepithelial tissue in a mammal in need thereof. The method comprises administering to the mammal a therapeutically effective amount of a compound of formula (I) or a pharmaceutically acceptable salt thereof.

In a third aspect, the present invention provides a method for the treatment of an astrocytic tumor in a mammal in need thereof. The method comprises administering to the mammal a therapeutically effective amount of a compound of formula (I) or a pharmaceutically acceptable salt thereof.

In a fourth aspect, the present invention provides a method for the treatment of glioblastoma multiforme in a mammal in need thereof. The method comprises administering to the mammal a therapeutically effective amount of a compound of formula (I) or a pharmaceutically acceptable salt thereof.

In a fifth aspect, the present invention provides a method for the treatment of a metastases in the central nervous system, from a tumor originating outside of the central nervous system, in a mammal in need thereof. The method comprises administering to the mammal a therapeutically effective amount of a compound of formula (I) or a pharmaceutically acceptable salt thereof.

In a sixth aspect, the present invention provides the use of a compound of formula (I) or a pharmaceutically acceptable salt thereof for the preparation of a medicament for the treatment of a primary CNS tumor in a mammal in need thereof.

In a seventh aspect, the present invention provides the use of a compound of formula (I) or a pharmaceutically acceptable salt thereof for the preparation of a medicament for the treatment of a tumor of neuroepithelial tissue in a mammal in need thereof.

In an eighth aspect, the present invention provides the use of a compound of formula (I) or a pharmaceutically acceptable salt thereof for the preparation of a medicament for the treatment of an astrocytic tumor in a mammal in need thereof.

In another aspect, the present invention provides the use of a compound of formula (I) or a pharmaceutically acceptable salt thereof for the preparation of a medicament for the treatment of glioblastoma multiforme in a mammal in need thereof.

In another aspect, the present invention provides the use of a compound of formula (I) or a pharmaceutically acceptable salt thereof for the preparation of a medicament for the treatment of metastases in the central nervous system, from a tumor originating outside of the central nervous system, e.g., lung cancer metastases, breast cancer metastases, adenocarcinoma of unknown primary site metastases, melanoma metastases, renal cell cancer metastases, colon cancer metastases, sarcoma metastases, Wilm's tumor metastases, neuroblastoma metastases, germ cell tumor metastases, acute lymphoblastic leukemia metastases, high grade non-Hodgkin's lymphoma metastases and acute myeloid leukemia metastases, in a mammal in need thereof.

In another aspect, the present invention provides a pharmaceutical composition comprising a compound of formula (I) or a pharmaceutically acceptable salt thereof for use in the treatment of a primary CNS tumor in a mammal in need thereof.

In another aspect, the present invention provides a provides a pharmaceutical composition comprising a compound of formula (I) or a pharmaceutically acceptable salt thereof for use in the treatment of a metastases in the central nervous system, from a tumor originating outside of the central nervous system, e.g., lung cancer metastases, breast cancer metastases, adenocarcinoma of unknown primary site metastases, melanoma metastases, renal cell cancer metastases, colon cancer metastases, sarcoma metastases, Wilm's tumor metastases, neuroblastoma metastases, germ cell tumor metastases, acute lymphoblastic leukemia metastases, high grade non-Hodgkin's lymphoma metastases and acute myeloid leukemia metastases, in a mammal in need thereof.

These and other aspects of the invention are described in further detail in the description which follows.

### DETAILED DESCRIPTION OF THE INVENTION

As used herein, “compound of formula (I)” means compound having the following structural formula:

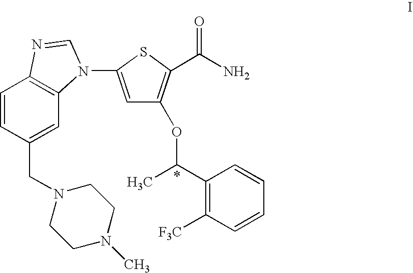

wherein * indicates the chiral carbon.

As used herein, “compound of formula (I-A)” means compound having the following structural formula:

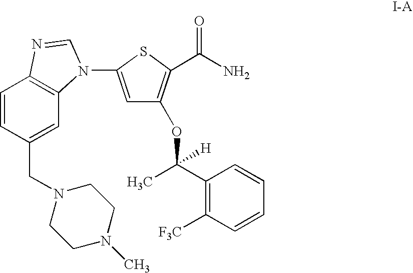

namely, 5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide.

As used herein, “compound of the invention” means a compound of formula (I) or (I-A) or a pharmaceutically acceptable salt thereof.

The terms “racemate” and “racemic mixture” as used herein refer to a mixture of the (R)- and the (S)-optical isomers (e.g., enantiomers) of the compound of formula (I) in equal, i.e. 50:50 proportion.

The term “enantiomerically enriched” as used herein refers to a preparation comprising a mixture of optical isomers in which the quantity of one enantiomer is higher than the quantity of the other. Thus, an “enantiomerically enriched” form of the compound of formula (I) comprises greater than 50% by weight of one enantiomer relative to the other. For example enantiomerically enriched 5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide refers to a composition comprising greater than 50% by weight of the (R)-enantiomer relative to the (S)-enantiomer of the compound. In one embodiment, the enantiomerically enriched compound of formula (I) comprises at least 75% by weight of one enantiomer relative to the other. In another embodiment, the enantiomerically enriched compound of formula (I) comprises at least 80% by weight of one enantiomer relative to the other. In one particular embodiment, an enantiomerically enriched compound comprises at least 85% by weight of one enantiomer relative to the other.

The term “enantiomerically pure” as used herein refers to enantiomerically enriched compounds comprising at least 90% by weight of one enantiomer relative to the other. In one embodiment, an enantiomerically pure compound comprises at least 95% by weight of one enantiomer relative to the other. In one particular embodiment, an enantiomerically pure compound comprises at least 99% by weight of one enantiomer relative to the other.

The compound of the invention may be utilized as a free base or as pharmaceutically acceptable salt. The pharmaceutically acceptable salts of the compound of the invention (or the enantiomerically enriched or pure forms thereof) include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic (mesylate), naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compound of the invention and its pharmaceutically acceptable salts. Specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts.

Processes for preparing pharmaceutically acceptable salts of the compound of the invention are conventional in the art. See, e.g., Burger's Medicinal Chemistry and Drug Discovery 5th Edition, Vol 1: Principles And Practice.

PLK is essential for cellular mitosis and accordingly, the compound of the invention is currently believed to be useful in the treatment of primary and secondary CNS tumors by inhibiting mitosis. “Inhibiting mitosis” refers to inhibiting the entry into the M phase of the cell cycle, inhibiting the normal progression of the M phase of the cell cycle once M phase has been entered and inhibiting the normal exit from the M phase of the cell cycle. The compound of the invention is an inhibitor of PLK. That is, the compound exhibits a pIC₅₀ greater than 7 in the PLK Inhibition assay described below in the examples, and an IC₅₀ less than 1 μM in the Methylene Blue, Cell-Titre Glo Growth or Multiple Cell Line cellular proliferation assays described in the examples below. Thus, the compound of the present invention may inhibit mitosis by inhibiting the cell's entry into mitosis, by inhibiting the cell's progression through mitosis or by inhibiting the cell's exit from mitosis. Inhibition of cellular proliferation was observed in a variety of systemic cancer cell lines tested in the Methylene Blue, Cell-Titre Glo Growth or Multiple Cell Line cellular proliferation assays. The data are presented in the examples below. Inhibition of cellular proliferation was also observed in a variety of glioma cell lines tested in the Multiple Cell Line cellular proliferation assay. The data are presented in the examples below.

It has now been discovered that the compound of the invention is capable of penetrating the central nervous system and crossing the intact blood-brain barrier. A Quantitative Whole Body Autoradiagraphy (QWBA) study was conducted to evaluate the quantitative tissue distribution of drug-related material following a single intravenous (IV) dose of radioactively-labeled compound of the invention (¹⁴C-labeled compound) to male Long-Evans (pigmented) rats. The method employed is described below in the examples. The concentrations of radioactivity observed in the brain tissue indicate that the ¹⁴C-labeled compound penetrated the intact blood-brain barrier. The observations that the compound of the invention inhibits a variety of solid and haematological cancer cells, coupled with the observation that the compound crosses the BBB supports the conclusion that the compound of the invention is reasonably expected to inhibit the proliferation of primary and secondary CNS tumors, including astrocytic tumors and metastases in the CNS from a variety of solid tumors and haematological malignancies originating outside of the CNS. M J van den Bent (2003) European J Cancer 39:2114-2120.

The present invention provides methods for the treatment of primary CNS tumors in a mammal (e.g., a human) in need thereof, which comprise the step of administering a therapeutically effective amount of a compound of the invention. Primary CNS tumors include:

tumors of neuroepithelial tissue such as:
astrocytic tumors including pilocytic, astrocytoma (diffuse, infiltrative, and fibrillary), anaplastic and glioblastoma multiforme;
oligodendroglial tumors and mixed gliomas such as oligodendroglioma well differentiated, anaplastic oligodendroglioma, mixed oligodendroglioma/astrocytoma and mixed anaplastic oligodendroglioma/anaplastic astrocytoma;
mixed gliomas;
ependymal tumors including myxopapillary ependymoma, ependymoma and anaplastic ependymoma;
choroid plexus tumors including choroid plexus papilloma and choroid plexus carcinoma;
neuronal and mixed neuronal-glial tumors including ganglioglioma, central neurocytoma, filum terminale paraganglioma and dysembryoplastic neuroepithelial tumor (DNET);
neuroblastic tumors;
pineal parenchymal tumors including pineocytoma and pineoblastoma; and
embryonal tumors including medulloblastoma, supratentorial primitive neuroectodermal tumor (PNET) and atypical teratoid/rhabdoid tumor;
   tumors of peripheral nerves such as:
schwannoma (neurinoma),
neurofibroma,
perineurioma and
malignant peripheral nerve sheath tumor (MPNST);
   tumors of the meninges (“menigeal tumors”) such as
tumors of meningothelial cells (including meningioma, atypical meningioma clear cell meningioma, chordoid meningioma, rhabdoid meningioma, papillary meningioma and anaplastic meningioma),
mesenchymal non-meningothelial tumors and
primary melanocytic lesions;
   lymphomas and haemopoietic neoplasms such as:
malignant lymphomas,
plasmacytoma and
granulocytic sarcoma;
   germ cell tumors such as:
germinoma,
embryonal carcinoma,
yolk sac tumor,
choriocarcinoma,
teratoma and mixed germ cell tumors; and
   tumors of the sellar region such as:
craniopharyngioma and
granular cell tumor.

The present invention provides methods of treatment and uses for each of the primary CNS tumors listed above, individually.

In one embodiment, the present invention provides methods for the treatment of a tumor of neuroepithelial tissue in a mammal (e.g., a human) in need thereof. In one embodiment, the present invention provides methods for the treatment of an astrocytic tumor in a mammal (e.g., a human) in need thereof. Astrocytic tumors include pilocytic astrocytoma, diffuse, infiltrative, and fibrillary astrocytoma, anaplastic astrocytoma and glioblastoma multiforme. In one particular embodiment, the present invention provides methods for the treatment of glioblastoma multiforme in a mammal (e.g., a human) in need thereof.

In one embodiment, the present invention provides methods for the treatment of a menigeal tumor (including those specific types identified above) in a mammal (e.g., a human) in need thereof. In one particular embodiment, the present invention provides methods for the treatment of a meningioma in a mammal (e.g., a human) in need thereof.

The present invention further provides a method for the treatment of secondary CNS tumors, i.e., metastases in the CNS, from a tumor originating outside of the CNS, in a mammal (e.g., human) in need thereof. As used herein, “metastasis in the CNS” refers to a tumor located in the CNS, which originated outside the CNS and has spread (metastasized) to the CNS. Tumors may metastasize from their original site to sites other than the CNS. The instant invention is directed toward treating those metastases occurring in the CNS. Specific metastases within the scope of the invention include lung cancer metastases, breast cancer metastases, adenocarcinoma of unknown primary site metastases, melanoma metastases, renal cell cancer metastases, colon cancer metastases, sarcoma metastases, Wilm's tumor metastases, neuroblastoma metastases, germ cell tumor metastases, acute lymphoblastic leukemia metastases, high grade non-Hodgkin's lymphoma metastases and acute myeloid leukemia metastases. While the foregoing represent primary tumors which are known to frequently metastasize to the CNS, it is expressly contemplated that the compound of the invention may be used to treat metastases in the CNS regardless of the origin of the primary tumor.

In one particular embodiment, the present invention provides a method for treating lung cancer metastases in the CNS in a mammal (e.g., human) in need thereof. In another particular embodiment, the present invention provides a method for treating breast cancer metastases in the CNS in a mammal (e.g., human) in need thereof. In another particular embodiment, the present invention provides a method for treating melanoma metastases in the CNS in a mammal (e.g., human) in need thereof. In another particular embodiment, the present invention provides a method for treating renal cell cancer metastases in the CNS in a mammal (e.g., human) in need thereof. In another particular embodiment, the present invention provides a method for treating colon cancer metastases in the CNS in a mammal (e.g., human) in need thereof. In another particular embodiment, the present invention provides a method for treating sarcoma metastases in the CNS in a mammal (e.g., human) in need thereof. In another particular embodiment, the present invention provides a method for treating neuroblastoma metastases in the CNS in a mammal (e.g., human) in need thereof. In another particular embodiment, the present invention provides a method for treating germ cell tumor metastases in the CNS in a mammal (e.g., human) in need thereof.

As used herein, the term “treatment” refers to alleviating the condition, eliminating or reducing the number of primary or secondary CNS tumor cells or the outward symptoms of the condition, slowing or eliminating the progression, invasion or metastatic spread of primary or secondary CNS tumor cells and preventing or delaying the recurrence of primary or secondary CNS tumors in a previously afflicted subject.

As used herein, the term “therapeutically effective amount” means an amount of a compound of the invention which is sufficient, in the mammal to which it is administered, to elicit the biological or medical response of a mammal (including a human) that is being sought, for instance, by a researcher or clinician. For example, a therapeutically effective amount of a compound of the invention for the treatment of primary CNS tumors is an amount sufficient to treat primary CNS tumors in the subject to which it is administered. A therapeutically effective amount of a compound of the invention for the treatment of metastases in the CNS from a tumor originating outside the CNS, is an amount sufficient to treat the tumor in the subject to which it is administered. In one embodiment of the present invention, a therapeutically effective amount of a compound of the invention is an amount sufficient to regulate, modulate, bind or inhibit PLK.

The precise therapeutically effective amount of a compound of the invention will depend on a number of factors including, but not limited to, the age and weight of the mammal being treated, the type and severity of the tumor requiring treatment, the nature of the formulation, and the route of administration, and will ultimately be at the discretion of the attendant physician or veterinarian. Typically, a compound of the invention will be given for treatment in the range of 0.1 to 200 mg/kg body weight of recipient (animal) per day or per dose or per cycle of treatment and more usually in the range of 1 to 100 mg/kg body weight per day or per dose or per cycle of treatment. Acceptable dosages may be from about 0.1 to about 2000 mg per day, dose, or cycle of treatment, and preferably from about 1 to about 1000 mg per day, dose, or cycle of treatment.

The compound of the invention can be used alone in the treatment of primary or secondary CNS tumors or can be used to provide additive or synergistic effects with certain existing chemotherapies and/or other anti-neoplastic therapies, e.g., cytotoxic chemotherapy, hormonal therapy, targeted kinase inhibitors, therapeutic monoclonal antibodies, surgery and radiation therapy. In addition, the compounds of the invention can be used to supplement the effectiveness of certain existing chemotherapies and/or other anti-neoplastic therapies. When used in combination with other anti-neoplastic therapies, the dose of the compound of the invention may be adjusted, based upon the knowledge and judgment of the skilled clinician.

The present invention also provides the use of the compound of the invention for the preparation of a medicament for the treatment of primary CNS tumors in a mammal (e.g., a human). The present invention further provides the use of a compound for the preparation of a medicament for the treatment of a tumor of neuroepithelial tissue in a mammal. The present invention further provides the use of a compound for the preparation of a medicament for the treatment of an astrocytic tumor in a mammal. In particular, the present invention provides the use of a compound for the preparation of a medicament for the treatment of a glioblastoma multiforme in a mammal. The present invention further provides the use of the compound of the invention for the preparation of a medicament for the treatment of secondary CNS tumors, in a mammal (e.g., human) in need thereof. Specific tumor metastases within the scope of the invention include lung cancer metastases, breast cancer metastases, adenocarcinoma of unknown primary site metastases, melanoma metastases, renal cell cancer metastases, colon cancer metastases, sarcoma metastases, Wilm's tumor metastases, neuroblastoma metastases, germ cell tumor metastases, acute lymphoblastic leukemia metastases, high grade non-Hodgkin's lymphoma metastases and acute myeloid leukemia metastases. In one particular embodiment, the present invention provides the use of the compound of the invention for the preparation of a medicament for the treatment of lung cancer metastases in the CNS in a mammal (e.g., human) in need thereof. In another particular embodiment, the present invention provides the use of the compound of the invention for the preparation of a medicament for the treatment of breast cancer metastases in the CNS in a mammal (e.g., human) in need thereof. In another particular embodiment, the present invention provides the use of the compound of the invention for the preparation of a medicament for the treatment of melanoma metastases in the CNS in a mammal (e.g., human) in need thereof. In another particular embodiment, the present invention provides the use of the compound of the invention for the preparation of a medicament for the treatment of renal cell cancer metastases in the CNS in a mammal (e.g., human) in need thereof. In another particular embodiment, the present invention provides the use of the compound of the invention for the preparation of a medicament for the treatment of colon cancer metastases in the CNS in a mammal (e.g., human) in need thereof. In another particular embodiment, the present invention provides the use of the compound of the invention for the preparation of a medicament for the treatment of sarcoma metastases in the CNS in a mammal (e.g., human) in need thereof. In another particular embodiment, the present invention provides the use of the compound of the invention for the preparation of a medicament for the treatment of neuroblastoma metastases in the CNS in a mammal (e.g., human) in need thereof. In another particular embodiment, the present invention provides the use of the compound of the invention for the preparation of a medicament for the treatment of germ cell tumor metastases in the CNS in a mammal (e.g., human) in need thereof.

While it is possible that, for use in the invention, a therapeutically effective amount of the compound of the invention may be administered as the raw chemical, it is typically presented as the active ingredient of a pharmaceutical composition or formulation. Pharmaceutical compositions comprising a compound of the invention are described in PCT Publication No. WO2007/030361, published 15 Mar. 2007 to GlaxoSmithKline.

The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. Preferably, the pharmaceutical composition is presented in unit dose form containing a predetermined amount of active ingredient per unit dose. Such a unit may contain a therapeutically effective dose of the compound of the invention or a fraction of a therapeutically effective dose such that multiple unit dosage forms might be administered at a given time to achieve the desired therapeutically effective dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art. Pharmaceutical formulations adapted for administration by oral (including buccal or sublingual), or parenteral (including subcutaneous, intramuscular, intravenous or intradermal) routes are preferred. In some instances, conventional agents for the treatment of primary and secondary CNS tumors have been administered via intra-thecal route (i.e., direct administration into the cerebral-spinal fluid), direct administration into the CNS (brain), such as via catheter following surgical resection of the tumor and intra-cavity deposition of a drug-infused wafer following surgical resection of the tumor. These conventional methods for administration of the compound of the invention, optionally in combination with one or more chemotherapeutic agents may be employed in the methods of the present invention as well.

As noted above, the compound of the invention may be employed alone in the methods of treating primary CNS tumors or in combination with one or more other compounds of the invention or in combination with other anti-neoplastic therapies. In particular, combination with other chemotherapeutic agents is envisaged as well as combination with surgical therapy and radiation therapy. The term “chemotherapeutic” as used herein refers to any chemical agent having a therapeutic effect on the subject to which it is administered. “Chemotherapeutic” agents include but are not limited to anti-neoplastic agents, analgesics and anti-emetics. As used herein, “anti-neoplastic agents” include both cytostatic and cytotoxic agents such as but not limited to cytotoxic chemotherapy, hormonal therapy, targeted kinase inhibitors and therapeutic monoclonal antibodies. Combination therapies according to the present invention thus comprise the administration of the compound of the invention and the use of at least one other cancer treatment method. In one particular embodiment, the present invention provides methods of treatment comprising administration of the compound of the invention in combination with radiation therapy. In particular, each dose of the compound of the invention may be administered before radiation therapy, concurrently with radiation therapy or after radiation therapy. In one embodiment, combination therapies according to the present invention comprise the administration of the compound of the invention and at least one other chemotherapeutic agent. In one particular embodiment, the present invention comprises the administration of the compound of the invention and at least one anti-neoplastic agent. As an additional aspect, the present invention provides the methods of treatment and uses as described above, which comprise administering the compound of the invention together with at least one chemotherapeutic agent. In one particular embodiment, the chemotherapeutic agent is an anti-neoplastic agent. In another embodiment, the present invention provides a pharmaceutical composition as described above further comprising at least one other chemotherapeutic agent, more particularly, the chemotherapeutic agent is an anti-neoplastic agent.

Typically, any chemotherapeutic agent that has activity against the tumor being treated may be utilized in combination with the compound of the invention, provided that the particular agent is clinically compatible with therapy employing a compound of the invention. Typical anti-neoplastic agents useful in such combinations include, but are not limited to, anti-microtubule agents such as diterpenoids and vinca alkaloids; platinum coordination complexes; alkylating agents such as nitrogen mustards (e.g., temozolomide), oxazaphosphor-ines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins (e.g., irinotecan); hormones and hormonal analogues; signal transduction pathway inhibitors; non-receptor tyrosine kinase angiogenesis inhibitors; immunotherapeutic agents; proapoptotic agents; and cell cycle signaling inhibitors. The compound of the invention may be used in combination with any one or more of the foregoing types of chemotherapeutic agents. Combination therapies of the compound of the invention together with another chemotherapeutic agent are described in PCT Publication No. 2007/030361, published 15 Mar. 2007 to GlaxoSmithKline. In one embodiment, the compound of the invention is employed in combination with a nitrogen mustard such as temozolamide. In one embodiment the compound of the invention is employed in combination with a topoisomerase I inhibition such as irinotecan. In one embodiment, the compound of the invention is employed in combination with an angiogenesis inhibitor such as a non-tryosine kinase angiogenesis inhibitor.

The compound of the invention may be prepared using the methods described in the example below and by the general methods described in PCT Publication No. WO2004/014899 to GlaxoSmithKline.

The following examples are provided for the purpose of further illustrating the invention and are not limiting thereof. The invention is defined solely by the claims which follow.

### EXAMPLES

The following abbreviations, as employed in the examples, have the recited meanings.

| | | | |
|---|---|---|---|
| g | gram(s) | H₂ | hydrogen |
| mg | milligram(s) | H₂O | water |
| mol | mole(s) | HCl | hydrochloric acid |
| mmol | millimole(s) | K₂CO₃ | potassium carbonate |
| N | normal | KOH | potassium hydroxide |
| L | liter(s) | MeOH | methanol |
| mL | milliliter(s) | MgSO₄ | magnesium sulfate |
| μL | microliter(s) | NaHCO₃ | sodium bicarbonate |
| h | hour(s) | Na₂SO₄ | sodium sulfate |
| min | minute(s) | NEt₃ | triethylamine |
| ° C. | degrees Centigrade | N₂ | nitrogen |
| Cs₂CO₃ | cesium carbonate | Pd/C | palladium on carbon |
| DCM | dichloromethane | THF | tetrahydrofuran |
| EtOAc | ethyl acetate | m | molar |
| XANTPHOS | (4,5-Bis(diphenyl- | HPLC | High Performance |
| | phosphino)-9,9- | | Liquid Chromato- |
| | dimethylxanthene) | | graphy |
| | is a commercilaly | | |
| | available catalyst | | |
| | from Aldrich | | |

Reagents are commercially available or are prepared according to procedures in the literature. In the following structures, “Me” refers to the group —CH₃.

### Intermediate Example 1

### Methyl 5-(6-bromo-1H-benzimidazol-1-yl)-3-hydroxy-2-thiophenecarboxylate

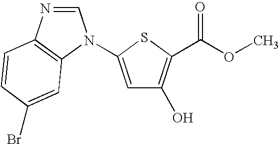

### Step A—Methyl 5-(6-bromo-1H-benzimidazol-1-yl)-3-{[(1, 1-dimethylethyl)(dimethyl)silyl]oxy}-2-thiophenecarboxylate

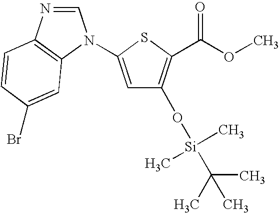

To a mixture of 5-bromo-1H-benzimidazole (43.78 g, 222.0 mmol) in chloroform (800 mL) was added N-methylimidazole (44.5 mL, 560.0 mmol), followed by methyl 2-chloro-3-oxo-2,3-dihydro-2-thiophenecarboxylate (44.8 g, 233.0 mmol). The reaction was stirred 20 h at room temperature, then N-methylimidazole (18.0 mL, 226.0 mmol) was added, followed by t-butyldimethylsilylchloride (36.8 g, 245.0 mmol). The reaction was stirred 1 hr, then quenched with MeOH and poured into DCM and water. The aqueous layer was extracted with DCM (3×). The combined organics were then dried over Na₂SO₄, concentrated and chromatographed on silica gel, eluting with a 50-to-75% gradient of 25% EtOAc in hexane/hexane to give 25.18 g (24%) of the title compound. MS (ESI): 467 [M+H]⁺.

### Step B—Methyl 5-(6-bromo-1H-benzimidazol-1-yl)-3-hydroxy-2-thiophenecarboxylate

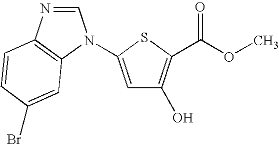

To a stirred solution of methyl 5-(6-bromo-1H-benzimidazol-1-yl)-3-{[(1,1-dimethylethyl)(dimethyl)silyl]oxy}-2-thiophenecarboxylate (25.18 g, 53.9 mmol) in THF (540.0 mL) was added 1.0M tetrabutylammonium fluoride in THF (60.0 mL, 60.0 mmol). The reaction was stirred 1.5 h then aqueous saturated NH₄Cl was added (200 mL). The resulting slurry was stirred 15 min then diluted with water (750 mL) and EtOAc (1.0 L). The aqueous layer was separated and its pH adjusted to 3.0 by addition of aqueous 1M HCl. The aqueous layer was then extracted with EtOAc (3×). The combined organic layers were washed with aqueous 0.1M HCl, brine, dried over MgSO₄ and concentrated under vacuum to give 19.4 g (100%) of the title compound as a light yellow solid. MS (ESI): 353 [M+H]⁺.

### Example 1

### 5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide

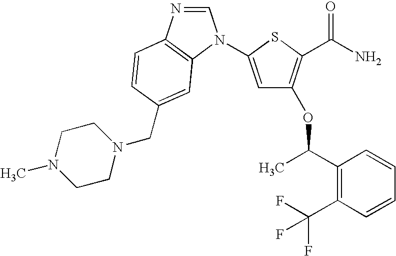

### Route 1:

### Step A—3-Methyl 5-(6-bromo-1H-benzimidazol-1-yl)-3-{(1R)-1-[2-(trifluoromethyl)phenyl]ethoxy}thiophene-2-carboxylate

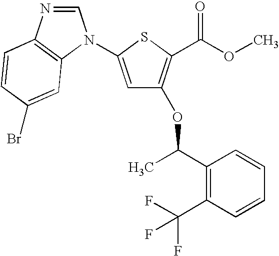

A slurry of polymer-supported triphenylphosphine (53.0 g, 2.04 mmol/g, 108 mmol), (1S)-1-[2-(trifluoromethyl)phenyl]ethanol (15.4 g, 81.0 mmol), and methyl 5-(6-bromo-1H-benzimidazol-1-yl)-3-hydroxythiophene-2-carboxylate (Intermediate Example 1) (19.0 g, 53.9 mmol) in DCM (750 mL) was stirred at room temperature, for 10 min. The slurry was then treated with di-tert-butyl azodicarboxylate (24.8 g, 108 mmol). The reaction mixture was stirred for 3 h, then poured through filter paper, washing the resin solids with DCM and MeOH. The filtrate was concentrated under vacuum and purified by silica gel chromatography, eluting with a 5-to-50% gradient of EtOAc/hexane to give 23.8 g (84%) of the title compound as a light yellow solid. ¹H NMR (400 MHz, DMSO-d₆): δ 8.63 (s, 1H), 7.97 (d, 1H, J=7.87 Hz), 7.80-7.71 (m, 3H), 7.65 (d, 1H, J=1.65 Hz), 7.57-7.48 (m, 2H), 7.35 (s, 1H), 5.99 (q, 1H, J=5.98 Hz), 3.83 (s, 3H), 1.65 (d, 3H, J=6.04 Hz); MS (ESI): 525 [M+H]⁺.

### Step B—Methyl 3-{(1R)-1-[2-(trifluoromethyl)phenyl]ethoxy}-5-(6-vinyl-1H-benzimidazol-1-yl)thiophene-2-carboxylate

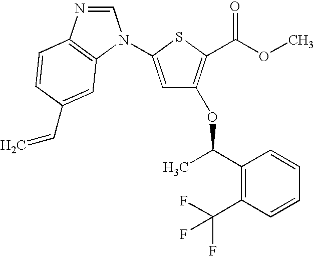

To a mixture of 3-methyl-5-(6-bromo-1H-benzimidazol-1-yl)-3-{(1R)-1-[2-(trifluoromethyl)phenyl]ethoxy}thiophene-2-carboxylate (23.8 g, 45.4 mmol), potassium vinyltrifluoroborate (7.25 g, 54.5 mmol) and triethylamine (6.3 mL, 45.4 mmol), stirred at room temperature in n-propanol (230 mL) was added [1,1′-bis(diphenylphosphino)-ferrocene]dichloropalladium(II) dichloromethane complex (750 mg, 0.91 mmol). The mixture was then heated to reflux and stirred for 3 h, then cooled to room temperature, poured into water and extracted with EtOAc (3×). The combined organic layers were washed with brine, dried over MgSO₄, concentrated under vacuum and purified by silica gel chromatography, eluting with a 10-to-40% gradient of EtOAc/hexane to give 17.06 g (80%) of the title compound as a yellow foam solid. ¹H NMR (400 MHz, DMSO-d₆): δ 8.59 (s, 1H), 7.98 (d, 1H, J=7.87 Hz), 7.80-7.71 (m, 3H), 7.59 (s, 1H), 7.56-7.52 (m, 2H), 7.39 (s, 1H), 6.85 (dd, 1H, J=10.98 and 17.75 Hz), 6.00 (q, 1H, J=6.10 Hz), 5.86 (d, 1H, J=17.56 Hz), 5.31 (d, 1H, J=10.98 Hz), 3.83 (s, 3H), 1.65 (d, 3H, J=6.04 Hz); MS (ESI): 473 [M+H]⁺.

### Step C—Methyl 5-[6-(1,2-dihydroxyethyl)-1H-benzimidazol-1-yl]-3-{(1R)-1-[2-(trifluoromethyl)phenyl]ethoxy}thiophene-2-carboxylate

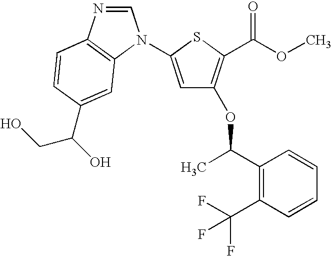

To a stirred solution of methyl 3-{(1R)-1-[2-(trifluoromethyl)phenyl]ethoxy}-5-(6-vinyl-1H-benzimidazol-1-yl)thiophene-2-carboxylate (17.06 g, 36.1 mmol) in 360 mL of acetone/water (3:1) was added 4-methylmorpholine N-oxide (5.1 g, 43.4 mmol) followed by 2.5 weight % solution osmium tetroxide in 2-methyl-2-propanol (10.0 mL, 0.8 mmol). The reaction was stirred at room temperature for 18 h, then quenched with aqueous (saturated) sodium sulfite. The mixture was extracted with EtOAc (3×). The combined organic layers were washed with brine, dried over MgSO₄, concentrated under vacuum and purified by silica gel chromatography, eluting with a 1-to-8% gradient of MeOH/DCM with 1% ammonium hydroxide to give 16.72 g (92%) of the title compound as a light yellow foam solid. ¹H NMR (400 MHz, DMSO-d₆): δ 8.59 (d, 1H, J=1.46 Hz), 7.98 (d, 1H, J=7.87 Hz), 7.80-7.68 (m, 3H), 7.59-7.52 (m, 2H), 7.36-7.31 (m, 2H), 5.95 (q, 1H, J=6.10 Hz), 5.37 (t, 1H, J=3.66 Hz), 4.76-4.64 (m, 2H), 3.83 (s, 3H), 3.46-3.42 (m, 2H), 1.65 (d, 3H, J=6.04 Hz); MS (ESI): 507 [M+H]⁺.

### Step D—Methyl 5-(6-formyl-1H-benzimidazol-1-yl)-3-{(1R)-1-[2-(trifluoromethyl)phenyl]ethoxy}thiophene-2-carboxylate

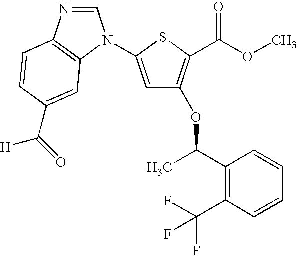

To a solution of methyl 5-[6-(1,2-dihydroxyethyl)-1H-benzimidazol-1-yl]-3-{(1R)-1-[2-(trifluoromethyl)phenyl]ethoxy}thiophene-2-carboxylate (16.72 g, 33.0 mmol) in 1:1:1 DCM/water/MeOH (220 mL) was added sodium periodate (10.58 g, 49.5 mmol). The resulting slurry was stirred for 1 h, then was diluted with water and EtOAc. The aqueous layer was extracted with EtOAc (3×). The combined organic layers were washed with brine, dried over MgSO₄ and concentrated under vacuum to give 14.76 g (94%) of the title compound as a light yellow foam solid. ¹H NMR (400 MHz, DMSO-d₆): δ 10.09 (s, 1H), 8.87 (s, 1H), 8.19 (s, 1H), 8.02-7.89 (m, 3H), 7.81-7.72 (m, 2H), 7.57-7.51 (m, 2H), 5.98 (q, 1H, J=6.10 Hz), 3.84 (s, 3H), 1.66 (d, 3H, J=6.22 Hz); MS (ESI): 475 [M+H]⁺.

### Step E—Methyl 5-{6-[(4-methylpiperazin-1-yl)methyl]-1H-benzimidazol-1-yl}-3-{(1R)-1-[2-(trifluoromethyl)phenyl]ethoxy}thiophene-2-carboxylate

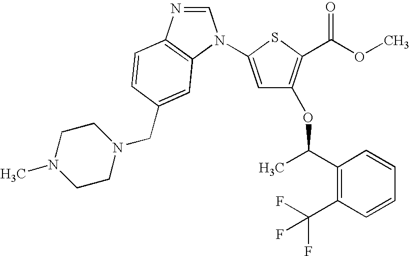

To a stirred solution of methyl 5-(6-formyl-1H-benzimidazol-1-yl)-3-{(1R)-1-[2-(trifluoromethyl)phenyl]ethoxy}thiophene-2-carboxylate (14.76 g, 31.1 mmol), n-methylpiperazine (5.72 mL, 62.3 mmol) and acetic acid (2.1 mL, 37.4 mmol) in dichloroethane (150 mL) was added sodium triacetoxyborohydride (9.9 g, 46.7 mmol). The reaction was stirred for 1.5 h, then aqueous 5% K₂CO₃ was added until the pH was approx. 8. The mixture was then diluted with EtOAc and water. The aqueous layer was extracted with EtOAc (3×). The combined organic layers were washed with brine, dried over Na₂SO₄, concentrated under vacuum and purified by silica gel chromatography, eluting with a 1-to-8% gradient of MeOH/DCM with 1% ammonium hydroxide to give 15.82 g (91%) of the title compound as a light yellow foam solid. ¹H NMR (400 MHz, DMSO-d₆): δ 8.57 (s, 1H), 7.98 (d, 1H, J=7.87 Hz), 7.80-7.68 (m, 3H), 7.54 (t, 1H, J=7.59 Hz), 7.46 (s, 1H), 7.33-7.28 (m, 2H), 5.97 (q, 1H, J=6.16 Hz), 3.83 (s, 3H), 3.55 (s, 2H), 2.45-2.20 (m, 8H), 2.13 (s, 3H), 1.66 (d, 3H, J=6.04 Hz); MS (ESI): 559 [M+H]⁺.

### Step F—5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide

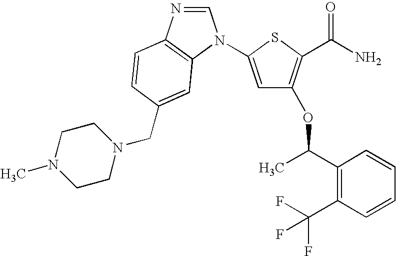

A mixture of methyl 5-{6-[(4-methylpiperazin-1-yl)methyl]-1H-benzimidazol-1-yl}-3-{(1R)-1-[2-(trifluoromethyl)phenyl]ethoxy}thiophene-2-carboxylate (15.82 g, 28.35 mmol) and 7N ammonia in MeOH (250 mL, 1.75 mol) was added to a high-pressure glass reaction flask. The flask was sealed, then heated to 80° C. for approx. 40 h. The flask was cooled to room temperature, opened, and the reaction mixture concentrated under vacuum, then purified by silica gel chromatography, eluting with a 2-to-8% gradient of MeOH/DCM with 1% ammonium hydroxide to give 14.11 g (92%) of the title compound as a white foam solid. ¹H NMR (400 MHz, DMSO-d₆): δ 8.49 (s, 1H), 7.93 (d, 1H, J=7.87 Hz), 7.86 (br s, 1H), 7.80-7.75 (m, 2H), 7.68 (d, 1H, J=8.23 Hz), 7.56 (t, 1H, J=7.68 Hz), 7.33 (s, 1H), 7.28 (d, 1H, J=8.42 Hz), 7.15 (br s, 1H), 7.06 (s, 1H), 5.94 (q, 1H, J=6.10 Hz), 3.52 (s, 2H), 2.45-2.20 (m, 8H), 2.13 (s, 3H), 1.74 (d, 3H, J=6.22 Hz); MS (ESI): 544 [M+H]⁺.

### Route 2:

### Step A—2-bromo-4-{[(methyloxy)methyl]oxy}-1-nitrobenzene

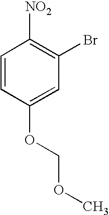

A solution of 3-bromo-4-nitrophenol (20.0 g, 91.7 mmol) in DCM (475 mL) was stirred at 0° C. Diisopropylethylamine (19.2 mL, 110.0 mmol) was added, followed by the drop wise addition of a solution of chloromethyl methyl ether (7.7 mL, 100.9 mmol) in DCM (25 mL). The reaction was stirred at 0° C. for 1 hr, then warmed to room temperature and quenched with water (150 mL). The mixture was poured into brine (150 mL) and the aqueous layer extracted with EtOAc (3×). The combined organic layers were washed with brine, dried over MgSO₄, concentrated under vacuum and chromatographed on silica gel (330 g), eluting with a 0-to-25% gradient of EtOAc/hexane to give 20.0 g (83%) of the title compound as a clear orange oil. ¹H NMR (400 MHz, DMSO-d₆): δ 8.07 (d, 1H, J=8.97 Hz), 7.50 (d, 1H, J=2.20 Hz), 7.21 (dd, 1H, J=8.97 and 2.38 Hz), 5.34 (s, 2H), 3.39 (s, 3H).

### Step B—Methyl 5-[(5-{[(methyloxy)methyl]oxy}-2-nitrophenyl)amino]-3-({(1R)-1-[2-(trifluoromethyl)phenyl]ethyl}oxy)-2-thiophenecarboxylate

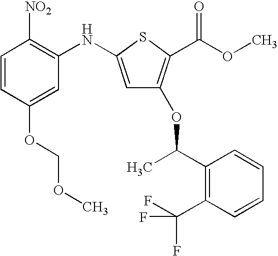

To a stirred solution of methyl 15-amino-3-({(1R)-1-[2-(trifluoromethyl)phenyl]-ethyl}oxy)-2-thiophenecarboxylate (1.32 g, 3.82 mmol) and 2-bromo-4-{[(methyloxy)methyl]oxy}-1-nitrobenzene (1.0 g, 3.82 mmol) in dioxane (20 mL) was added tris(dibenzylideneacetone)dipalladium(0) (70.0 mg, 0.076 mmol) and XANTPHOS (97.0 mg, 0.17 mmol) followed by cesium carbonate (6.2 g, 19.0 mmol). The mixture was heated to 60° C. and stirred for 12 h, then cooled to room temperature, diluted with EtOAc and filtered through Celite, washing the solids with EtOAc and DCM. The filtrate was concentrated under vacuum and chromatographed on silica gel (120 g), eluting with a 5-to-35% gradient of EtOAc/hexane to give 1.64 g (82%) of the title compound as a red oil. ¹H NMR (400 MHz, DMSO-d₆): δ 9.85 (s, 1H), 8.12 (d, 1H, J=9.33 Hz), 7.90 (d, 1H, J=7.87 Hz), 7.74 (m, 2H), 7.53 (t, 1H, J=7.68 Hz), 6.84 (d, 1H, J=2.56 Hz), 6.73-6.68 (m, 2H), 5.77-5.72 (m, 1H), 5.23 (s, 2H), 3.75 (s, 3H), 3.37 (s, 3H), 1.58 (d, 3H, J=6.22 Hz); MS (ESI): 527 [M+H]⁺.

### Step C—Methyl 5-(6-{[(methyloxy)methyl]oxy}-1H-benzimidazol-1-yl)-3-({(1R)-1-[2-(trifluoromethyl)phenyl]ethyl}oxy)-2-thiophenecarboxylate

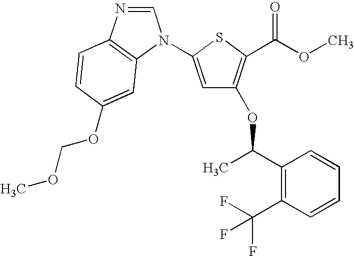

To a high-pressure hydrogenation reaction flask was added methyl 5-[(5-{[(methyloxy)methyl]oxy}-2-nitrophenyl)amino]-3-({(1R)-1-[2-(trifluoromethyl)phenyl]ethyl}oxy)-2-thiophenecarboxylate (2.0 g, 3.8 mmol), pyridinium p-toluene sulfonate (95.0 mg, 0.38 mmol), 5% by weight platinum on carbon (sulfided) (740 mg, 0.19 mmol) and trimethylorthoformate (40 mL). The flask was purged with N₂ (gas) vacuum (3×), then with H₂ (gas)/vacuum (3×). H₂ gas was then applied at 50 psi for 3 h. The reaction mixture was then filtered through Celite, washing the solids with EtOAc and DCM. The filtrate was then concentrated under vacuum to 1.92 g (100%) of the title compound as a light yellow foam solid. ¹H NMR (400 MHz, DMSO-d₆): δ 8.49 (s, 1H), 7.98 (d, 1H, J=8.05 Hz), 7.79-7.66 (m, 3H), 7.53 (t, 1H, J=7.59 Hz), 7.35 (s, 1H), 7.22 (d, 1H, J=2.20 Hz), 7.06 (dd, 1H, J=8.78 and 2.20 Hz) 5.97 (q, 1H, J=6.04 Hz), 5.23 (s, 2H), 3.83 (s, 3H), 3.39 (s, 2H), 1.65 (d, 3H, J=6.22 Hz); MS (ESI): 507 [M+H]⁺.

### Step D—Methyl 5-(6-hydroxy-1H-benzimidazol-1-yl)-3-({(1R)-1-[2-(trifluoromethyl)phenyl]ethyl}oxy)-2-thiophenecarboxylate

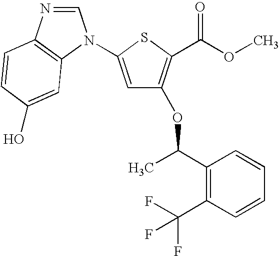

To a stirred solution of methyl 5-(6-{[(methyloxy)methyl]oxy}-1H-benzimidazol-1-yl)-3-({(1R)-1-[2-(trifluoromethyl)phenyl]ethyl}oxy)-2-thiophenecarboxylate (8.18 g, a different batch using procedure analogous to Example 1, Route 2, Step C, 16.16 mmol) in 1:1 THF/MeOH (130 mL) was added 1N HCl in water (65 mL, 65.0 mmol). The reaction mixture was heated to 35° C. and stirred for 72 h then cooled to room temperature. The reaction was poured into DCM (500 mL) and water added (100 mL). The mixture was neutralized to pH 7 by addition of aqueous (saturated) NaHCO₃. The aqueous layer was then extracted with DCM (1×) and EtOAc (1×). The combined organic layers were dried over MgSO₄, concentrated under vacuum, and chromatographed on silica gel (120 g), eluting with a 10-to-60% gradient of EtOAc/hexane to give 6.9 g (92%) of the title compound as a light salmon colored foam solid. ¹H NMR (400 MHz, DMSO-d₆): δ 9.62 (s, 1H), 8.41 (s, 1H), 8.00 (d, 1H, J=7.87 Hz), 7.80-7.71 (m, 2H), 7.56-7.51 (m, 2H), 7.38 (s, 1H), 7.05 (d, 1H, J=2.01 Hz), 6.81 (dd, 1H, J=8.70 and 2.11 Hz) 5.95 (m, 1H), 3.83 (s, 3H), 1.64 (d, 3H, J=6.23 Hz); MS (ESI): 463 [M+H]⁺.

### Step E—Methyl 3-({(1R)-1-[2-(trifluoromethyl)phenyl]ethyl}oxy)-5-(6-{[(trifluoromethyl)sulfonyl]oxy}-1H-benzimidazol-1-yl)-2-thiophenecarboxylate

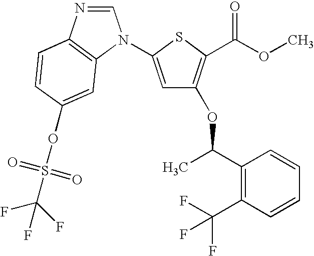

To a stirred, cooled (0° C.) solution of methyl 5-(6-hydroxy-1H-benzimidazol-1-yl)-3-({(1R)-1-[2-(trifluoromethyl)phenyl]ethyl}oxy)-2-thiophenecarboxylate (2.49 g, 5.38 mmol) and n-phenyltrifluoromethane sulfonamide (2.06 g, 5.76 mmol) in DCM (30 mL) was added diisopropylethylamine (2.0 mL, 11.5 mmol). The reaction was allowed to warm to room temperature and stirred for 12 h. The reaction mixture was then concentrated under vacuum, and chromatographed on silica gel (120 g), eluting with a 5-to-40% gradient of EtOAc/hexane to give 3.12 g (98%) of the title compound as a light yellow solid. ¹H NMR (400 MHz, DMSO-d₆): δ 8.76 (s, 1H), 8.01-7.94 (m, 2H), 7.80-7.70 (m, 3H), 7.56-7.43 (m, 3H), 5.98 (q, 1H, J=6.10 Hz), 3.84 (s, 3H), 1.65 (d, 3H, J=6.22 Hz); MS (ESI): 595 [M+H]⁺.

### Step F—3-{(1R)-1-[2-(Trifluoromethyl)phenyl]ethoxy}-5-(6-vinyl-1H-benzimidazol-1-yl)thiophene-2-carboxylate

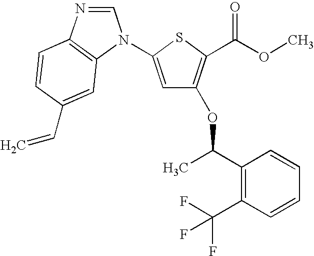

To a mixture of methyl 3-({(1R)-1-[2-(trifluoromethyl)phenyl]ethyl}oxy)-5-(6-{[(trifluoromethyl)sulfonyl]oxy}-1H-benzimidazol-1-yl)-2-thiophenecarboxylate (20.69 g, from a different batch using procedure analogous to Example 1, Route 2, Step E, 34.83 mmol), potassium vinyltrifluoroborate (5.6 g, 42.10 mmol) and triethylamine (4.85 mL, 34.86 mmol), stirred at room temperature in n-propanol (175 mL) was added [1,1′-bis(diphenylphosphino)-ferrocene]dichloropalladium(II) dichloromethane complex (570 mg, 0.70 mmol). The mixture was then heated to reflux and stirred for 3 h, then cooled to room temperature, poured into water and extracted with EtOAc (3×). The combined organic layers were washed with brine, dried over MgSO₄, concentrated under vacuum and purified by silica gel chromatography, eluting with a 10-to-50% gradient of EtOAc/hexane to give 12.98 g (79%) of the title compound as a light yellow foam solid. MS (ESI): 473 [M+H]⁺.

### Step G—Methyl 5-[6-(1,2-dihydroxyethyl)-1H-benzimidazol-1-yl]-3-{(1R)-1-[2-(trifluoromethyl)phenyl]ethoxy}thiophene-2-carboxylate

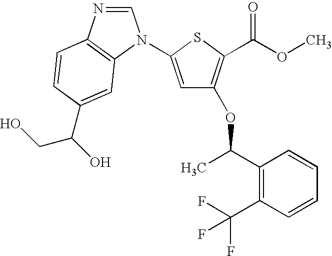

The title compound can be prepared by a procedure analogous to Example 1, Route 1, Step C.

### Step H—Methyl 5-(6-formyl-1H-benzimidazol-1-yl)-3-{(1R)-1-[2-(trifluoromethyl)phenyl]ethoxy}thiophene-2-carboxylate

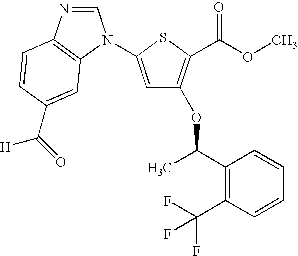

The title compound can be prepared by a procedure analogous to Example 1, Route 1, Step D.

### Step 1—Methyl 5-{6-[(4-methylpiperazin-1-yl)methyl]-1H-benzimidazol-1-yl}-3-{(1R)-1-[2-(trifluoromethyl)phenyl]ethoxy}thiophene-2-carboxylate

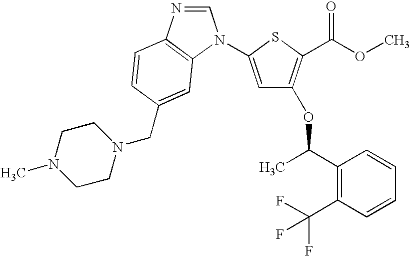

The title compound can be prepared by a procedure analogous to Example 1, Route 1, Step E.

### Step J—5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide

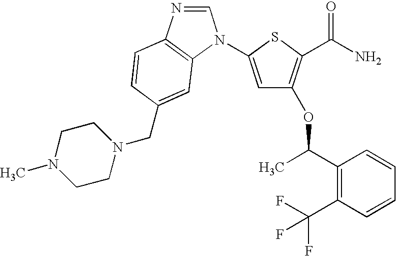

The title compound can be prepared by a procedure analogous to Example 1, Route 1, Step F.

### Route 3:

### Step A—Methyl 5-{6-[(4-methylpiperazin-1-yl)methyl]-1H-benzimidazol-1-yl}-3-({(1R)-1-[2-(trifluoromethyl)phenyl]ethyl}oxy)thiophene-2-carboxylate

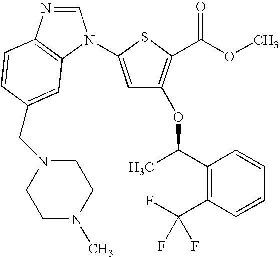

To a stirred, solution of methyl 5-[6-(chloromethyl)-1H-benzimidazol-1-yl]-3-({(1R)-1-[2-(trifluoromethyl)phenyl]ethyl}oxy)thiophene-2-carboxylate (150 mg, 0.30 mmol) in dioxane (1.0 mL) was added N-methylpiperazine (50 μL, 0.45 mmol). The reaction was heated at 60° C. for 18 h, cooled to room temperature and concentrated under vacuum. The residue was dissolved in EtOAc and water. The aqueous layer was extracted with EtOAc. The combined organic layers were washed with brine, dried over sodium sulfate, concentrated under vacuum, and purified by silica gel chromatography eluting with a gradient of 0-to-10% MeOH/DCM, with 1% ammonium hydroxide, to give 134 mg (79%) of the title compound as a white solid. MS (ESI): 559 [M+H]⁺.

### Step B—5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide

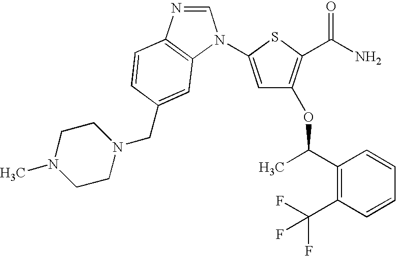

The title compound can be prepared by a procedure analogous to Example 1, Route 1, Step F.

### Route 4:

### Step A—4-[Bis(methyloxy)methyl]-2-bromo-1-nitrobenzene

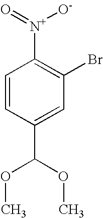

A solution of 3-bromo-4-nitrobenzaldehyde (7.97 g, 34.6 mmol), which was prepared in a manner analogous to the literature procedure (Katritzky, A. R.; Xie, L. Tetrahedron Letters 1996, 37, 347-350), trimethyl orthoformate (11.4 mL, 104 mmol), and p-toluenesulfonic acid hydrate (329 mg, 1.73 mmol) in MeOH (69 mL) was refluxed for 3 h. The reaction was then quenched by addition of saturated aqueous ammonium hydroxide (1 mL) and concentrated onto silica gel. Purification by column chromatography (10 to 25% EtOAc:hexanes) provided 8.76 g (92%) of the title compound as an orange oil. ¹H NMR (300 MHz, CDCl₃): δ 7.89 (m, 2H), 7.59 (m, 1H), 5.47 (s, 1H), 3.38 (s, 6H).

### Step B—Methyl 5-({5-[bis(methyloxy)methyl]-2-nitrophenyl}amino)-3-({(1R)-1-[2-(trifluoromethyl)phenyl]ethyl}oxy)-2-thiophenecarboxylate

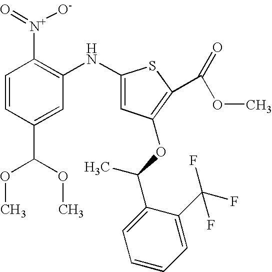

A solution of tris(dibenzylideneacetone) dipalladium(0) (117 mg, 0.127 mmol), 9,9-dimethyl-4,5-bis(diphenylphosphino)xanthene (162 mg, 0.280 mmol), methyl 5-amino-3-({(1R)-1-[2-(trifluoromethyl)phenyl]ethyl}oxy)-2-thiophenecarboxylate (2.31 g, 6.69 mmol), 4-[bis(methyloxy)methyl]-2-bromo-1-nitrobenzene (1.76 g, 6.37 mmol), and cesium carbonate (10.39 g, 31.89 mmol) in 1,4-dioxane (25 mL) was prepared in a round-bottom flask under N₂. The flask was evacuated and refilled three times with N₂ and then stirred at 60° C. for 16 h. The reaction mixture was then diluted with tetrahydrofuran (100 mL) and concentrated onto silica gel. Purification by column chromatography (5 to 75% EtOAc:hexanes) provided 2.79 g (81%) of the title compound as a red foam. ¹H NMR (300 MHz, CDCl₃): δ 9.63 (br s, 1H), 8.21 (m, 1H), 7.94 (m, 1H), 7.62 (m, 2H), 7.48 (s, 1H), 7.40 (m, 1H), 7.02 (m, 1H), 6.47 (s, 1H), 5.73 (q, 1H, J=6.2 Hz), 3.88 (s, 3H), 3.34 (s, 1H), 3.31 (s, 3H), 3.28 (s, 3H), 1.72 (d, 3H, J=6.2 Hz); MS (ESI): 541 [M+H]⁺.

### Step C—Methyl 5-{6-[bis(methyloxy)methyl]-1H-benzimidazol-1-yl}-3-({(1R)-1-[2-(trifluoromethyl)phenyl]ethyl}oxy)-2-thiophenecarboxylate

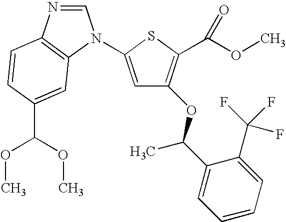

To a solution of methyl 5-({5-[bis(methyloxy)methyl]-2-nitrophenyl}amino)-3-({(1R)-1-[2-(trifluoromethyl)phenyl]ethyl}oxy)-2-thiophenecarboxylate (2.71 g, 5.01 mmol) in trimethyl orthoformate (50 mL) in a Fischer-Porter bottle was added pyridinium p-toluenesulfonate (126 mg, 0.501 mmol) and sulfided platinum on carbon (5 wt % Pt, 977 mg, 0.250 mmol Pt). The mixture was hydrogenated on a Fischer-Porter hydrogenation apparatus at 50 psi of hydrogen until the uptake of hydrogen had ceased (17 h). The reaction mixture was filtered through a sintered glass filter to remove the catalyst, washing with DCM (75 mL). The eluant was concentrated to afford 2.61 g (100%) of the crude title compound as an orange oil, which was carried on to the next step without purification. MS (ESI): 521 [M+H]⁺.

### Step D—Methyl 5-(6-formyl-1H-benzimidazol-1-yl)-3-({(1R)-1-[2-(trifluoromethyl)phenyl]ethyl}oxy)-2-thiophenecarboxylate

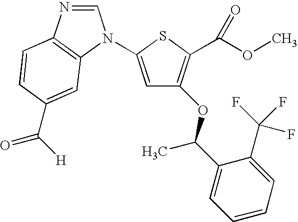

To a solution of crude methyl 5-{6-[bis(methyloxy)methyl]-1H-benzimidazol-1-yl}-3-({(1R)-1-[2-(trifluoromethyl)phenyl]ethyl}oxy)-2-thiophenecarboxylate (2.61 g, 5.01 mmol) (from Step C, above) in acetone (20 mL) and water (5 mL) was added pyridinium p-toluenesulfonate (126 mg, 0.501 mmol). The reaction stirred for 2 h at room temperature and was then poured into water (30 mL) and saturated aqueous NaHCO₃ (30 mL). The mixture was extracted with DCM (2×30 mL). The combined organic fractions were dried over sodium sulfate, filtered, and concentrated onto silica gel. Purification by column chromatography (30 to 100% EtOAc:hexanes) provided 1.37 g (58%, 2 steps) of the title compound as a light yellow solid. ¹H NMR (300 MHz, CDCl₃): δ 10.06 (s, 1H), 8.13 (s, 1H), 7.96-7.88 (m, 4H), 7.72-7.61 (m, 2H), 7.44 (m, 1H), 6.82 (s, 1H), 5.84 (q, 1H, J=6.3 Hz), 3.95 (s, 3H), 1.79 (d, 3H, J=6.3 Hz); MS (ESI): 475 [M+H]⁺.

### Step E—Methyl 5-{6-[(4-methyl-1-piperazinyl)methyl]-1H-benzimidazol-1-yl}-3-({(1R)-1-[2-(trifluoromethyl)phenyl]ethyl}oxy)-2-thiophenecarboxylate

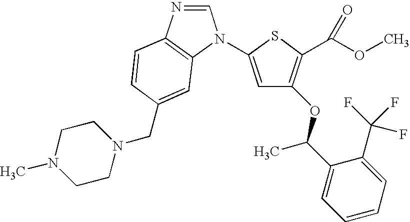

The title compound can be prepared by a procedure analogous to Example 1, Route 1, Step E.

### Step F—5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide

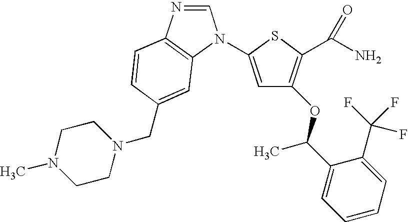

The title compound can be prepared by a procedure analogous to Example 1, Route 1, Step F.

### Route 5:

### Step A—1-[(3-bromo-4-nitrophenyl)methyl]-4-methylpiperazine

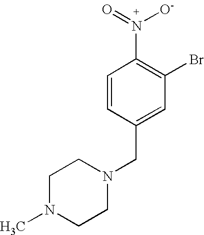

N-methyl piperazine (0.95 mL, 8.56 mmol) was added dropwise to a suspension of 3-bromo-4-nitrobenzaldehyde (0.985 g, 4.28 mmol) and acetic acid (0.29 mL, 5.14 mmol) in toluene (5.5 mL) at room temperature. The reaction mixture was stirred for 1 h at room temperature before the addition of sodium triacetoxyborohydride (1.36 g, 6.42 mmol). The reaction mixture was stirred for 2 h. Further toluene (2.1 mL) and sodium triacetoxyborohydride (0.18 g, 0.86 mmol) was added and the reaction mixture was stirred for 2 hours and then quenched by the addition of MeOH (0.99 mL). The reaction mixture was stirred for 30 min, saturated aqueous NaHCO₃ (6.01 mL) was then added and the reaction mixture was stirred overnight. The phases were separated and the aqueous layer was extracted with toluene (4.0 mL). The combined organic extracts were concentrated under reduced pressure and purified by flash column chromatography (EtOAc:MeOH:NEt₃ 9:1:0.1) to give the title compound (1.18 g, 88%) as a low melting point waxy brown solid.

δ_{H} (400 MHz, CDCl₃) 7.81 (1H, d, J 8.3, Ar H), 7.74 (1H, d, J 1.5, Ar H), 7.42 (1H, dd, J 1.7, 8.3, Ar H), 3.53 (2H, s, NCH₂Ar), 2.49 (8H, br s, 2×NCH₂CH₂), 2.31 (3H, s, NCH₃).

### Step B—1-[(3-fluoro-4-nitrophenyl)methyl]-4-methylpiperazine

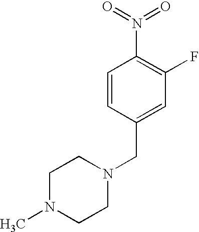

N-methyl piperazine (1.97 mL, 17.8 mmol) was added dropwise to a solution of 3-fluoro-4-nitrobenzaldehyde (1.50 g, 8.88 mmol) and acetic acid (0.20 mL, 3.54 mmol) in toluene (8.4 mL) at room temperature. The reaction mixture was stirred for 1.5 h at room temperature before the addition of further toluene (3.15 mL) and sodium triacetoxyborohydride (2.97 g, 14.0 mmol). The reaction mixture was stirred for 70 min and then further sodium triacetoxyborohydride (0.40 g, 1.9 mmol) was added. The reaction mixture was stirred for 50 min, and then quenched by the addition of MeOH (2 mL) and saturated aqueous NaHCO₃ (10 mL). The reaction mixture was stirred for 30 min. The phases were separated and the aqueous layer was extracted with toluene (2×20 mL, 1×10 mL). The combined organic extracts were concentrated under reduced pressure and purified by flash column chromatography (EtOAc:MeOH:NEt₃ 19:1:0.1) to give the title compound (1.85 g, 82%) as an orange oil.

δ_{H} (400 MHz, CDCl₃) 8.02 (1H, t, J 8.1, Ar H), 7.34 (1H, d, J 11.9, Ar H), 7.26 (1H, d, J 8.6, Ar H), 3.57 (2H, s, NCH₂Ar), 2.49 (8H, br s, 2×NCH₂CH₂), 2.31 (3H, s, NCH₃); LRMS found m/z [ES⁺] 254.

### Step C—1-[(3-chloro-4-nitrophenyl)methyl]-4-methylpiperazine

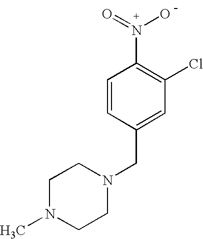

N-methyl piperazine (2.50 mL, 22.5 mmol) was added dropwise to a solution of 3-chloro-4-nitrobenzaldehyde (2.09 g, 11.3 mmol) and acetic acid (0.29 mL, 5.14 mmol) in toluene (11.7 mL) at room temperature. The reaction mixture was stirred for 1.5 h at room temperature before the addition of further toluene (4.4 mL) and sodium triacetoxyborohydride (3.58 g, 16.9 mmol). The reaction mixture was stirred at room temperature overnight and then quenched by the addition of MeOH (3 mL) and saturated aqueous NaHCO₃ (23 mL). The reaction mixture was stirred for 30 min. The phases were separated and the aqueous layer was extracted with toluene (2×30 mL). The combined organic extracts were concentrated under reduced pressure and purified by flash column chromatography (EtOAc:MeOH:NEt₃ 9:1:0.1) to give the title compound (2.17 g, 71%) as an orange oil.

δ_{H} (400 MHz, CDCl₃) 7.84 (1H, t, J 8.3, Ar H), 7.56 (1H, d, J 1.5, Ar H), 7.38 (1H, dd, J 8.3, 1.5, Ar H), 3.54 (2H, s, NCH₂Ar), 2.48 (8H, br s, 2×NCH₂CH₂), 2.30 (3H, s, NCH₃).

### Step D—Methyl 5-({5-[(4-methyl-1-piperazinyl)methyl]-2-nitrophenyl}amino)-3-({(1R)-1-[2-(trifluoromethyl)phenyl]ethyl}oxy)-2-thiophenecarboxylate

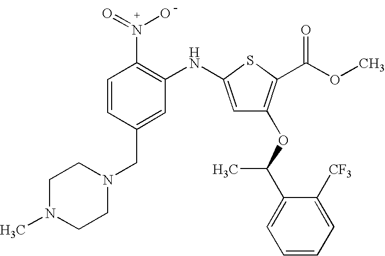

A mixture of 1-[(3-bromo-4-nitrophenyl)methyl]-4-methylpiperazine (0.675 g, 2.15 mmol), methyl 5-amino-3-({(1R)-1-[2-(trifluoromethyl)phenyl]ethyl}oxy)-2-thiophenecarboxylate (0.794 g, 2.30 mmol), Cs₂CO₃ (3.50 g, 10.7 mmol), XANTPHOS (0.053 g, 0.088 mmol) and tris(benzylideneacetone)dipalladium(0) (0.039 g, 0.043 mmol) in dioxane (5.4 mL) was heated to 55° C. for 1.5 h, analysed by HPLC, then cooled to room temperature. Heptane (2.4 mL), charcoal (0.27 g) and celite (0.27 g) were added. The suspension was stirred for 30 min at room temperature, and then filtered over celite, rinsing with toluene (13.5 mL×3). The filtrate was concentrated under reduced pressure and purified by flash column chromatography (DCM:MeOH:NEt₃ 9:1:0.1) to give the title compound (0.867 g, 70%) as a red amorphous solid.

δ_{H} (400 MHz, CDCl₃) 9.72 (1H, s, NH), 8.14 (1H, d, J 8.8, Ar H), 7.93 (1H, d, J 8.8, Ar H), 7.67-7.57 (2H, m, Ar H), 7.44-7.35 (2H, m, Ar H), 6.92 (1H, dd, J 8.8, 1.2, Ar H), 6.43 (1H, s, SC═CH), 5.73 (1H, q, J 6.4, CH₃CHO), 3.88 (3H, s, OCH₃), 3.47 and 3.41 (2×1H d, J 14.5, NCH₂Ar), 2.48 (8H, br s, 2×NCH₂CH₂), 2.30 (3H, s, NCH₃), 1.72 (3H, d, J 6.4, CH₃CHO); LRMS found m/z [ES⁻] 577.

Alternatively, methyl 5-({5-[(4-methyl-1-piperazinyl)methyl]-2-nitrophenyl}amino)-3-({(1R)-1-[2-(trifluoromethyl)phenyl]ethyl}oxy)-2-thiophenecarboxylate may be prepared by either of the following methods:

A mixture of 1-[(3-chloro-4-nitrophenyl)methyl]-4-methylpiperazine (0.313 g, 1.16 mmol), methyl 5-amino-3-({(1R)-1-[2-(trifluoromethyl)phenyl]ethyl}oxy)-2-thiophenecarboxylate (0.401 g, 1.16 mmol), K₂CO₃ (0.401 g, 2.90 mmol), X-Phos (0.0587 g, 0.12 mmol), tris(benzylideneacetone)dipalladium(0) (0.0531 g, 0.06 mmol) and tert-butanol (4.6 mL) in a sealed tube was heated at 80° C. for 18 h. The crude reaction mixture was filtered through celite, washing with toluene and then purified by flash column chromatography (EtOAc:MeOH:NEt₃) to give the title compound (0.534 g, 70%) as a red amorphous solid.

A solution of 1-[(3-fluoro-4-nitrophenyl)methyl]-4-methylpiperazine (0.733 g, 2.90 mmol) in acetonitrile (5 mL) was added dropwise to a mixture of methyl 5-amino-3-({(1R)-1-[2-(trifluoromethyl)phenyl]ethyl}oxy)-2-thiophenecarboxylate (1.00 g, 2.90 mmol) and KOH (0.325 g, 5.79 mmol) in acetonitrile (10 mL) at 0° C. The reaction mixture was allowed to warm to room temperature overnight and then heated to 40° C. for 1 h until analysis by HPLC showed complete consumption of starting materials. The reaction mixture was partitioned between EtOAc (50 mL) and H₂O (50 mL). The organic layer was washed with saturated aqueous NaHCO₃ (10 mL), concentrated under reduced pressure and then purified by flash column chromatography (DCM:MeOH 98:2 to 85:15, then EtOAC:MeOH 99:1 to 85:15) to give the title compound (0.85 g, 50%) as a red amorphous solid.

### Step E—Methyl 5-({2-amino-5-[(4-methyl-1-piperazinyl)methyl]phenyl}amino)-3-({(1R)-1-[2-(trifluoromethyl)phenyl]ethyl}oxy)-2-thiophenecarboxylate

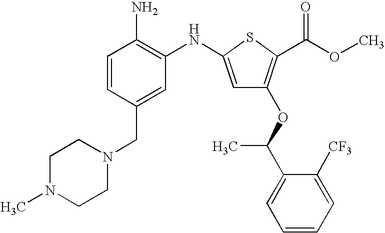

A mixture of methyl 5-({5-[(4-methyl-1-piperazinyl)methyl]-2-nitrophenyl}amino)-3-({(1R)-1-[2-(trifluoromethyl)phenyl]ethyl}oxy)-2-thiophenecarboxylate (0.24 g, 0.415 mmol), 5% Pd/C (0.144 g), HCl (1.25 M in MeOH, 0.034 mL, 0.043 mmol), trimethylorthoformate (1.66 mL, 15.2 mmol) in toluene (0.58 mL) was hydrogenated under a H₂ atmosphere at 1.1 bar pressure overnight. The vessel was purged with N₂ and the reaction mixture was analysed by HPLC to confirm presence of the title compound which was not purified, but used crude in the next step. LRMS found m/z [ES⁺] 549.

### Step F—Methyl 5-{6-[(4-methyl-1-piperazinyl)methyl]-1H-benzimidazol-1-yl}-3-({(1R)-1-[2-(trifluoromethyl)phenyl]ethyl}oxy)-2-thiophenecarboxylate

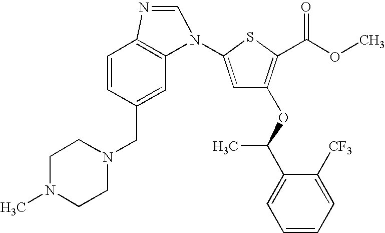

HCl (1.25 M in MeOH, 1.00 mL, 1.25 mmol) was added to a crude solution of methyl 5-({2-amino-5-[(4-methyl-1-piperazinyl)methyl]phenyl}amino)-3-({(1R)-1-[2-(trifluoromethyl)phenyl]ethyl}oxy)-2-thiophenecarboxylate from the previous step (2.5 mL). The reaction mixture was stirred for 10 min and then filtered through celite, washing with toluene (30 mL). The filtrate was concentrated under reduced pressure and partitioned between EtOAc (15 mL) and saturated aqueous Na₂CO₃ (15 mL) and then purified by flash column chromatography (EtOAc:MeOH:NEt₃ 9:1:0.1) to give the title compound (0.175 g, 75%) as a pale yellow amorphous solid.

δ_{H} (400 MHz, CDCl₃) 7.96-7.88 (2H, m, Ar H), 7.74 (1H, d, J 8.3, Ar H), 7.70-7.58 (2H, m, Ar H), 7.46-7.32 (3H, m, Ar H), 6.76 (1H, s, SC═CH), 5.83 (1H, q, J 6.1, CH₃CHO), 3.93 (3H, s, OCH₃), 3.62 and 3.58 (2×1H d, J 13.0, NCH₂Ar), 2.50 (8H, br s, 2×NCH₂CH₂), 2.31 (3H, s, NCH₃), 1.78 (3H, d, J 6.1, CH₃CHO); LRMS found m/z [ES⁻] 559.

### Step G—5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide

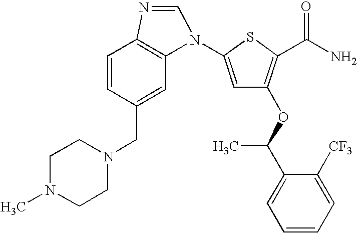

Formamide (1.075 mL, 26.5 mmol) followed by 25% w/w sodium methoxide in MeOH (1.82 mL, 7.6 mmol) are added to a solution of methyl 5-{6-[(4-methyl-1-piperazinyl)methyl]-1H-benzimidazol-1-yl}-3-({(1R)-1-[2-(trifluoromethyl)phenyl]ethyl}oxy)-2-thiophenecarboxylate (5.0 g, 8.95 mmol) in THF (50 mL) and toluene (10 mL) at room temperature. The reaction mixture is heated at ca 65° C. for about 18 h and then cooled to ca 30° C. The reaction mixture is diluted with EtOAc (25 mL) and H₂O (25 mL) and then the biphasic mixture is separated. The organic phase is washed sequentially with H₂O (25 ml), saturated aqueous Na₂CO₃ and finally H₂O (2×25 mL). The organic phase is then concentrated by rotary evaporation and the concentrate diluted with EtOAc (30 mL) and then heated at ca 70° C. for about 1 h. The resulting suspension is then cooled to about 20° C. and stirred at this temperature for ca 18 h. The suspension is then stirred at 0-5° C. for 2 h and the product, isolated by filtration, washed with EtOAc (5 mL) and dried under vacuum at ca 25° C. to constant weight (2.93 g, 60.2%).

δ_{H} (400 MHz, CDCl₃) ¹H NMR (400 MHz, DMSO-d₆): δ 8.49 (s, 1H), 7.93 (d, 1H, J=7.87 Hz), 7.86 (br s, 1H), 7.80-7.75 (m, 2H), 7.68 (d, 1H, J=8.23 Hz), 7.56 (t, 1H, J=7.68 Hz), 7.33 (s, 1H), 7.28 (d, 1H, J=8.42 Hz), 7.15 (br s, 1H), 7.06 (s, 1H), 5.94 (q, 1H, J=6.10 Hz), 3.52 (s, 2H), 2.45-2.20 (m, 8H), 2.13 (s, 3H), 1.74 (d, 3H, J=6.22 Hz); MS (ESI): 544 [M+H]⁺.

### Pharmaceutical Composition Example

The compound of the invention may be formulated as a 20 mg/mL sterile solution for intravenous injection. The sterile solution may be formulated by dissolving the compound of invention in glacial acetic acid using Water for Injection as a diluent (Composition A). The sterile solution may be formulated by dissolving the compound of invention in glacial acetic acid followed by the addition of Sulfobutyl ether beta-cyclodextrin (Captisol®) and using Water for Injection as a diluent (Composition B).

The solution is sterilized by filtration and typically diluted to the desired volume prior to administration using Dextrose Injection USP (5%).

Typical concentrations are provided in the Table below:

| | Composition A | Composition B |
|---|---|---|
| Component | (mg/mL) | (mg/mL) |
| Compound of the Invention | 20.0 | 20.0 |
| Glacial Acetic Acid | 3.0 | 3.0 |
| Water for Injection | q.s. | q.s. |
| Sulfobutyl ether | — | 300 |
| beta-cyclodextrin | | |

The compound of invention may be formulated at a sufficient concentration (5 to 50 mg/mL) as a sterile solution for intravenous injection. The formulation may contain buffers to achieve and maintain a desired pH. The buffers may include Acetate, Citrate, Tartrate, Phosphate, Carbonate and Trimethamine (Tris) in concentration of 10-50 mM. The formulation may contain solubilizers to improve solubility of the compound of invention. Solubilizers may include Sulfobutyl ether beta-cyclodextrin (Captisol®), Poly (ethylene) Glycol 300/400, Propylene Glycol, Ethanol, Hydroxypropyl β-Cyclodextrin.

The solution may be terminally sterilized by filtration or by autoclaving. The solution is typically diluted to the desired volume prior to administration using Dextrose Injection USP (5%).

### Biological Examples

### I. Quantitative Tissue Distribution of Drug-Related Material Using Whole-Body Autoradiography (QWBA)

The study evaluated the quantitative tissue distribution of drug-related material following a single intravenous (IV) dose of radioactively labeled compound of the invention (herein after “¹⁴C-labeled compound”) to male Long-Evans (pigmented) rats, using whole-body autoradiography.

A. ¹⁴C-labeled Compound Dose Solution Preparation

On the day of dosing, the ¹⁴C-labeled compound IV dose solution was prepared at a target concentration of 3.125 mg/mL (50 μCi/mL) by dissolving appropriate quantities of non-radiolabeled compound of the invention and ¹⁴C-labeled compound in 10% sulfobutylether beta cyclodextrin with acetate buffer, and 5% dextrose for injection USP. The solution was protected from light and stirred continuously on a magnetic stir plate at ambient temperature throughout the dosing procedure. Triplicate (1 top, 1 middle and 1 bottom of vial) pre-dose and post-dose aliquots (0.1 mL) of the ¹⁴C-labeled compound dose solution were diluted in 10 mL of dimethyl sulfoxide. Aliquots (0.1 mL) of the diluted dose solution were counted for 10 min using liquid scintillation counting (LSC) to check homogeneity and concentration of ¹⁴C-labeled compound in the dose solution. The stability of ¹⁴C-labeled compound in the dose solution for the duration of dosing was determined using radio-high performance liquid chromatography (radio-HPLC) by analyzing duplicate pre- and post-dose aliquots of the dose solution. The residual dose formulation was stored at approximately −70° C.

B. Dosing in Animals

Seven partially pigmented, male Long-Evans rats, with a mean body weight of 287.7 g and approximately 8 weeks old at the time of dosing received a single IV administration of the dose solution. All rats were maintained on Lab Diet 2005 Certified Rodent Diet, and filtered water from domestic supply was provided ad libitum. Animals were not fasted prior to dosing. Animals were kept under standard environmental conditions where the temperature and humidity ranges were set to maintain 65-75° F. and 20-70% respectively. Animal rooms were set to maintain at least 10 air exchanges per hour and the room lighting cycle was set to maintain a 12-h light/dark cycle.

Each rat was randomized and given a unique study-specific identification number, identified by tail-marking. Rats were individually housed in shoebox cages with wire mesh bottoms. Body weights were determined the morning prior to dosing. Each animal received a single nominal IV dose of approximately 12.5 mg/kg body weight at a dose volume of 4 mL/kg. Actual doses administered ranged from 12.55 to 13.57 mg/kg with a mean of 12.98 mg/kg.

C. Whole-Body Autoradiography

One rat at each time point 0.5 hour (h), 2 h, 6 h, 24 h, 3 days, 7 days and 35 days post-dose was deeply anesthetized, euthanized and frozen. The frozen carcasses were embedded in 2% carboxymethylcellulose and each block was mounted on the object stage of a cryomicrotome (Leica CM3600 Cryomacrocut, Nusslock, Germany) maintained at approximately −20° C. Sagittal sections, approximately 40 μm thick supported on adhesive tape, were taken from various levels through the block until samples of the selected tissues or biological matrices, along with QC standards, were obtained, where possible.

Sections were allowed to dry by sublimation in the cryomicrotome at −20° C. for at least 48 h. Sections were mounted on cardboard backing, covered with plastic wrapping and exposed along with 14C-spiked calibration standards to 14C-sensitive imaging plates (Molecular Dynamics, Sunnyvle, Calif.). The imaging plates and sections were enclosed in exposure cassettes and allowed to expose at room temperature for at least four days. At the end of the exposure time, the sections were removed from the imaging place. The plates were scanned and the images stored. Quantification, relative to the calibration standards was performed by image densitometry using MCID image analysis software (Imaging Research, St. Catherine's Ontario, Canada). Concentrations of radioactivity were expressed in terms of μg equivalents of compound per g sample (μg equiv/g).

D. Results and Conclusions

Concentrations of the radioactivity derived from the compound of the invention in various tissues are reported in Table 1.

TABLE 1

| | | Concentration of Radioactivity (μg equiv/g) | | | | | | |
|---|---|---|---|---|---|---|---|---|
| | | Time point | | | | | | |
| Tissue Type | Tissue | 0.5 h | 2 h | 6 h | 24 h | 3 d | 7 d | 35 d |
| Vascular/ | Aorta | 2.52 | 0.74 | 0.20 | BLQ | BLQ | BLQ | NS |
| Lymphatic | Blood (cardiac) | 2.40 | 0.73 | 0.18 | BLQ | BLQ | BLQ | NS |
| | Bone Marrow | 36.77 | 24.71 | 6.72 | 0.25 | 0.09 | 0.09 | NS |
| | Mandibular Lymph Nodes | 25.90 | 23.47 | 5.99 | 0.47 | 0.16 | 0.12 | NS |
| | Spleen | 46.05 | 32.54 | 7.54 | 0.52 | 0.18 | 0.16 | 0.06 |
| | Thymus | 15.10 | 13.32 | 5.30 | 0.36 | 0.08 | 0.07 | NS |
| Excretory/ | Bile (in duct) | 137.32 | 88.12 | 40.90 | 3.34 | 2.95 | 1.00 | NS |
| Metabolic | Kidney | 54.24 | 17.93 | 3.41 | 0.56 | 0.15 | 0.09 | NS |
| | Renal Cortex | 77.72 | 22.79 | 3.49 | 0.50 | 0.16 | 0.12 | NS |
| | Renal Medulla | 35.24 | 12.98 | 3.17 | 0.60 | 0.10 | 0.08 | NS |
| | Liver | 27.98 | 19.470 | 7.95 | 1.75 | 1.09 | 0.24 | 0.11 |
| CNS | Brain | 3.19 | 0.36 | 0.09 | 0.13 | 0.10 | 0.08 | NS |
| | Brain (striatum) | 4.28 | 0.52 | 0.23 | 0.29 | 0.30 | 0.14 | NS |
| | Choroid Plexus | 32.56 | 9.73 | 2.79 | 2.34 | 1.22 | 0.24 | NS |
| | Meninges | 10.31 | 1.24 | 1.24 | 4.88 | 14.11 | 5.34 | 5.09 |
| | Pineal Gland | 51.52 | 27.49 | 7.37 | 0.64 | NS | NS | NS |
| Endocrine | Adrenal Cortex | 69.75 | 19.96 | 4.81 | 0.40 | 0.15 | 0.10 | NS |
| | Adrenal Medulla | 31.05 | 9.27 | 2.68 | 0.60 | 0.14 | 0.13 | NS |
| | Pituitary Gland | 56.09 | 28.86 | 20.62 | 2.20 | 0.90 | 0.93 | NS |
| | Thyroid Gland | 183.79 | 67.62 | 8.28 | 0.60 | 0.35 | 0.25 | NS |
| Secretory | Lachrymal Gland (exorbital) | 35.55 | 28.07 | 10.44 | 3.49 | 0.16 | 0.13 | NS |
| | Harderian Gland | 21.66 | 21.83 | 21.01 | 12.69 | 12.14 | 1.28 | NS |
| | Lachrymal Gland (intraorbital) | 32.27 | 25.30 | 8.98 | 4.43 | 12.14 | 0.22 | NS |
| | Pancreas | 36.35 | 13.54 | 3.36 | 0.88 | 0.11 | BLQ | NS |
| | Salivary Gland | 40.70 | 26.09 | 8.91 | 0.25 | 0.05 | BLQ | NS |
| Adipose | Fat (brown) | 17.51 | 9.71 | 2.35 | 0.84 | 0.25 | 0.06 | NS |
| | Fat (abdominal) | 1.55 | 0.97 | 0.13 | 0.10 | BLQ | BLQ | NS |
| Dermal | Skin (non-pigmented) | 5.80 | 4.70 | 3.25 | 0.33 | 0.17 | BLQ | NS |
| | Skin (pigmented) | 5.58 | 6.84 | 3.54 | 1.05 | 1.69 | BLQ | BLQ |
| Reproductive | Epididymis | 2.24 | 1.99 | 0.87 | 1.29 | 0.88 | 0.38 | 0.12 |
| | Preputial Gland | 41.53 | 48.28 | 32.05 | 30.65 | 30.04 | 10.05 | NS |
| | Prostate Gland | 7.61 | 7.98 | 1.80 | 0.18 | NS | BLQ | NS |
| | Seminal Vesicles | 0.49 | 1.44 | 0.68 | BLQ | BLQ | BLQ | NS |
| | Testis | 0.96 | 0.95 | 0.83 | 1.19 | 0.51 | 0.28 | 0.27 |
| Muscular | Muscle (skeletal) | 21.87 | 3.39 | 0.78 | 0.19 | 0.06 | BLQ | NS |
| | Myocardium (heart) | 18.50 | 4.78 | 1.03 | 0.23 | 0.09 | 0.07 | NS |
| Respiratory | Lung | 42.37 | 14.03 | 4.45 | 0.35 | 0.08 | 0.08 | NS |
| Tract | Nasal Turbinates | 2.50 | 1.39 | 1.05 | 0.65 | 0.14 | 0.19 | NS |
| Alimentary | Cecum Mucosa | 19.42 | 16.97 | 63.28 | 1.42 | 0.06 | BLQ | NS |
| Canal | Esophagus | 4.97 | 6.86 | 2.98 | 1.0 | 0.10 | BLQ | NS |
| | Large Intestine Mucosa | 18.30 | 11.31 | 4.33 | 2.21 | 0.70 | 0.08 | NS |
| | Rectum Mucosa | 16.91 | 5.21 | 5.19 | 0.21 | 0.14 | BLQ | NS |
| | Small Intestine Mucosa | 27.82 | 39.98 | 6.46 | 0.62 | 0.07 | 0.08 | NS |
| | Stomach Mucosa | 37.74 | 9.39 | 1.73 | 0.65 | 0.16 | BLQ | NS |
| Ocular | Lens | BLQ | BLQ | BLQ | 0.23 | BLQ | 0.09 | NS |
| | Uveal Tract | 71.64 | 63.60 | 68.33 | 79.67 | 56.63 | 41.08 | 30.64 |

BLQ: Below the limit of quantitation (<0.05 μg equiv/mg)
NS: Not sampled (Tissue could not be visually identified because of non-detectable radioactivity).

The highest blood concentration of radioactivity occurred at 0.5 h post-dose (2.40) and dropped to <0.05 μg equiv/mg by 24 h post-dose. The concentrations of radioactivity measured in brain tissues protected by the blood-brain barrier (brain, brain striatum and meninges) were generally a their highest at 0.5 h and were similar to those observed in blood at early time points (0.5 h, 2 h and 6 h post-dose). Concentrations in brain tissue remained quantifiable up to 7 days post-dose. For the brain striatum, 1.3-3.4 times higher concentrations of radioactivity were observed, compared to other brain tissues.

The highest concentrations at 0.5 h post-dose were observed in the thyroid gland, renal cortex, uveal tract, adrenal cortex, pituitary gland and pineal gland. The concentration of radioactivity in blood was 2.40 μg equiv/mg at this timepoint, and most tissues tested were above this level.

At 7 days post-dose, tissue concentrations had dropped to close to or lower than 1 μg equiv/mg except for the uveal tract, preputial gland and meninges.

At 35 days post-dose, quantifiable tissue concentrations were still present in the meninges, uveal tract, testis, epididymis, liver and spleen.

The concentrations of radioactivity observed in the brain tissue indicate that the ¹⁴C-labeled compound penetrated the blood-brain barrier. There was some evidence for differential distribution of radioactivity within brain, with striatum region showing higher concentrations than remaining brain tissue.

### II. Assay for Inhibition of PLK1

A. Preparation of 6×N-Terminal His-Tagged PLK Kinase Domain

6× N-terminal His-tagged PLK kinase domain (amino acids 21-346 proceeded by MKKGHHHHHHD) SEQ ID: No. 1 was prepared from baculovirus infected T. ni cells under polyhedrin promoter control. All procedures were performed at 4° C. Cells were lysed in 50 mM HEPES, 200 mM NaCl, 50 mM imidazole, 5% glycerol; pH 7.5. The homogenate was centrifuged at 14K rpm in a SLA-1500 rotor for 1 h and the supernatant filtered through a 1.2 micron filter. The supernatant was loaded onto a Nickel chelating Sepharose (Amersham Pharmacia) column and washed with lysis buffer. Protein was eluted using 20%, 30% and 100% buffer B steps where buffer B is 50 mM HEPES, 200 mM NaCl, 300 mM imidazole, 5% glycerol; pH 7.5. Fractions containing PLK were determined by SDS-PAGE. Fractions containing PLK were diluted five-fold with 50 mM HEPES, 1 mM DTT, 5% glycerol; pH 7.5, then loaded on an SP Sepharose (Amersham Pharmacia) column. After washing the column with 50 mM HEPES, 1 mM DTT, 5% glycerol; pH 7.5, PLK was step eluted with 50 mM HEPES, 1 mM DTT, 500 mM NaCl; 5% glycerol; pH 7.5. PLK was concentrated using a 10 kDa molecular weight cutoff membrane and then loaded onto a Superdex 200 gel filtration (Amersham Pharmacia) column equilibrated in 25 mM HEPES, 1 mM DTT, 500 mM NaCl, 5% glycerol; pH 7.5. Fractions containing PLK were determined by SDS-PAGE. PLK was pooled, aliquoted and stored at −80° C. Samples were quality controlled using mass spectrometry, N-terminal sequencing and amino acid analysis.

B. Enzyme Activity +/−Inhibitors was Determined as Follows:

All measurements were obtained under conditions where signal production increased linearly with time and enzyme. Test compound was added to white 384-well assay plates (0.1 μL for 10 μL and some 20 μL assays, 1 μL for some 20 μL assays) at variable known concentrations in 100% DMSO. DMSO (1-5% final, as appropriate) and EDTA (65 mM in reaction) were used as controls. Reaction Mix was prepared as follows at 22° C.:
25 mM HEPES, pH 7.2
15 mM MgCl2
1 μM ATP
0.05 μCi/well ³³P-γ ATP (10 Ci/mMol)
1 μM substrate peptide (Biotin-Ahx-SFNDTLDFD) SEQ ID: No. 2
0.15 mg/mL BSA
1 mM DTT
2 nM PLK1 kinase domain (added last)

Reaction Mix (10 or 20 μL) was quickly added to each well immediately following addition of enzyme via automated liquid handlers and incubated 1-1.5 h at 22° C. The 20 μL enzymatic reactions were stopped with 50 μL of stop mix (50 mM EDTA, 4.0 mg/ml Streptavidin SPA beads in Standard Dulbecco's PBS (without Mg²⁺ and Ca²⁺), 50 μM ATP) per well. The 10 μL reactions were stopped with 10 μL of stop mix (50 mM EDTA, 3.0 mg/mL Streptavidin-coupled SPA Imaging Beads (“LeadSeeker”) in Standard Dulbecco's PBS (without Mg²⁺ and Ca²⁺), 50 μM ATP) per well. Plates were sealed with clear plastic seals, spun at 500×g for 1 min or settled overnight, and counted in Packard TopCount for 30 seconds/well (regular SPA) or imaged using a Viewlux imager (LeadSeeker SPA). Signal above background (EDTA controls) was converted to percent inhibition relative to that obtained in control (DMSO-only) wells.

C. Results

The data are reported in Table 2 below. In Table 1, +=pIC₅₀<6; ++=pIC₅₀ 6-8; +++=pIC₅₀>8.

### III. Methylene Blue Growth Inhibition Assay—Inhibition of Cell Proliferation by PLK1 Inhibitors

Generally, exponentially growing cell lines of different tumor origins, cultured in appropriate media containing 10% fetal bovine serum at 37° C. in a 5% CO₂ incubator were plated at low density (less than 2000 cells/well) in 96-well plates. Twenty four hours post-plating, cells were treated with different concentrations of compound ranging from 10 uM to 0.04 nM. Several wells were left untreated as a control. Seventy two hours post-treatment, cell numbers were determined using 100 μl per well of methylene blue (Sigma M9140) (0.5% in 50:50 Ethanol:water). Stain was incubated at room temperature for 30 minutes before plates were rinsed and dye solubilized in 1% N-lauroyl sarcosine, sodium salt, (Sigma L5125, in PBS) (further details of a methylene blue assay are described below). Plates were read on a microplate reader, measuring the OD at 620 nm.

Percent inhibition of cell growth was expressed as percent proliferation relative to 100% proliferation (control). Concentration of test compound that inhibited 50% of cell growth (IC₅₀) was determined by 4 parameter fit of data using XLfit, (value of no cell control was subtracted from all samples for background). The data are shown in Table 1 below and represent a compilation of several different experiments, each performed using the general parameters outlined above, although minor variations may have been employed in some instances.

In one assay, normal human foreskin fibroblasts (HFF), human colon (HCT116, RKO), lung (H460, A549) and breast (MCF7) tumor cell lines were cultured in high glucose DMEM (Life Technologies) containing 10% fetal bovine serum (FBS) at 37° C. in a humidified 5% CO₂, 95% air incubator. Cells were harvested using trypsin/EDTA, counted using a haemocytometer, and plated in 100 μL of culture media per well, at the following densities, in a 96-well tissue culture plate (Falcon 3075): HFF 5,000 cells/well, HCT116 3,000 cells/well, RKO 2,500 cells/well, H460 2,500 cells/well, A549 5,000 cells/well, MCF7 4,000 cells/well. The next day, compound was diluted in low glucose DMEM containing 100 μg/mL gentamicin, at twice the final required concentration, from 10 mM stock solutions in DMSO. 100 μL/well of these dilutions were added to the 100 μL of media currently in the assay plates. Medium containing 0.6% DMSO was added to control wells. The final concentration of DMSO in all wells was 0.3%. Cells were incubated at 37° C., 5% CO₂ for 72 h. Medium was removed by aspiration. Cell biomass was estimated by staining cells with 80 μL per well methylene blue (Sigma M9140, 0.5% in 50:50 ethanol:water), and incubation at room temperature for 30-60 min. Stain was removed by aspiration and the plates rinsed by immersion in water, then air-dried. To release stain from the cells, 100 μL of solubilization solution was added (1% N-lauroyl sarcosine, Sodium salt, Sigma L5125, in PBS), and plates were shaken gently for about 30 min. Optical density at 620 nM was measured on a microplate reader. Percent inhibition of cell growth was calculated relative to vehicle treated control wells. Concentration of compound that inhibits 50% of cell growth (IC₅₀) was interpolated using nonlinear regression (Levenberg-Marquardt) and the equation, y=Vₘₐₓ*(1−(x/(K+x)))+Y2, where “K” was equal to the IC₅₀.

The data are reported in Table 2 below. In Table 2, +=IC₅₀>1 μM; ++=IC₅₀ 0.1-1 μM: +++=IC₅₀<0.1 μM.

### IV. Determination of Protein Binding to Human Plasma Proteins Using Equilibrium Dialysis

96 Well Plate (High Throughput Dialysis): Stock solution of compound was spiked into human plasma at a target concentration of 2000 ng/mL. The mixture was inverted gently several times to insure homogeneity and triplicate 50 μL aliquots were collected to verify initial concentrations. Following assembly of dialysis plate (HTDialysis membrane strips, molecular weight cut off limit of 12,000-14,000 daltons), spiked plasma (150 μL) was placed in the donor compartment of the well and Phosphate Buffered Saline pH 7.4 (150 μL) in the receiver compartment. Eight wells were set up per compound and plasma type. Plate was placed in a 37° C. incubator on a plate shaker. Following the 6 h incubation period, the plate was removed. Single 50 μL aliquots from each donor and receiver compartment (per well) were analyzed.

Sample analysis was by LC/MS/MS (results reported as Drug Peak Area/Internal Standard Peak Area ratios). Protein binding assay can also be performed using dialysis cells instead of HT Dialysis 96 well plates. The data are reported in Table 1 below. In Table 1, % Protein Binding, +=>98%; ++=95-98%: +++=<95%.

### VI. High Throughput Solubility Assay

Two samples are prepared. One (the standard sample) contains the compound at a fixed concentration of 20 μM in an aqueous/organic mixed solvent cocktail. The other (the test sample) contains the compound at a maximum concentration of 200 μM in pH 7.4, 0.05M phosphate buffer and shaking for 24 h. The test sample is filtered by 0.45μ filter and then spun for 10 min to remove any undissolved solid. HPLC analyses are preformed on these samples. The peak areas are used for computing solubility. The data are reported in Table 2 below. In Table 2, solubility, +=<30 μM; ++=30-100 μM: +++=>100 μM.

TABLE 2

| | | | H460 | MCF7 | A549 | RKO | HFF | | |
|---|---|---|---|---|---|---|---|---|---|
| | PLK1 | HCT116 | IC₅₀ | IC₅₀ | IC₅₀ | IC₅₀ | IC₅₀ | % Protein | Solubility |
| Example # | pIC₅₀ | IC₅₀ (μM) | (μM) | (μM) | (μM) | (μM) | (μM) | Binding | (μM) |
| 1 | +++ | +++ | +++ | +++ | +++ | +++ | +++ | +++ | +++ |

Table 1 shows that the compound of the invention possess enzyme and cell potency in PLK1 enzyme assay and methylene blue cell proliferation assay in multiple cell lines examined. The compound of the invention is soluble in pH 7.4, 0.05 M phosphate buffer. The compound of the invention exhibits protein binding in human serum by equilibrium dialysis assay.

### VII. Cell-Titer-Glo—Inhibition of Cell Proliferation by PLK1 Inhibitors

Exponentially growing cell lines of different tumor origins, cultured in appropriate media containing 10% fetal bovine serum at 37° C. in a 5% CO₂ incubator were plated at low density (less than 2000 cells/well) in 96-well plates. Twenty four hours post-plating, cells were treated with different concentrations of compound ranging from 10 uM to 0.04 nM. Several wells were left untreated as a control. Seventy two hours post-treatment, cell numbers were determined using 50-100 ul per well of CellTiter-Glo (Promega #G7573). Plates were incubated at room temperature for 15 minutes and the chemiluminescent signal was read on the Victor V or Envison 2100 reader.

Percent inhibition of cell growth was expressed as percent proliferation relative to 100% proliferation (control). Concentration of compound that inhibited 50% of cell growth (IC₅₀) was determined by 4 parameter fit of data using XLfit, (value of no cell control was subtracted from all samples for background). The Cell-Titer Glo data are shown in Table 3 below and represent a compilation of several different experiments, each performed using the general parameters outlined above, although minor variations may have been employed in some instances. In Table 3, +=IC₅₀>1 μM; ++=IC₅₀ 0.5-1 μM: +++=IC₅₀<0.5 μM.

TABLE 3

| Cell Line | Tissue | Ex 1 |
|---|---|---|
| HCT116 IC₅₀ | colon | +++ |
| A549-L IC₅₀ | lung | +++ |
| COLO205 IC₅₀ | colon | +++ |
| HT29 IC₅₀ | colon | +++ |
| MX-1 IC₅₀ | breast | +++ |
| SKOV-3 IC₅₀ | ovary | +++ |
| LNCaP IC₅₀ | prostate | +++ |
| P388 IC₅₀ | blood | +++ |
| H1299 IC₅₀ | lung | +++ |
| Hela IC₅₀ | cervix | +++ |
| HN5 IC₅₀ | head and neck | +++ |
| MCF7 IC₅₀ | breast | +++ |
| MV522 IC₅₀ | lung | +++ |
| MDA-MB-468 IC₅₀ | breast | +++ |
| PANC-1 IC₅₀ | pancreas | +++ |
| MiaPaca IC₅₀ | pancreas | +++ |
| ASPC3 IC₅₀ | pancreas | +++ |
| BXPC3 IC₅₀ | pancreas | +++ |

### VIII. Multiple Cell Line Screen—Inhibition of Cell Proliferation by PLK1 Inhibitors

Growth properties for multiple cell lines were used to determine two seeding densities for each cell line that would be used in the screen. Cell lines were thawed and grown to 70-80% confluency before being used in the assay. On day −1, cells were seeded at 2 densities in 384-well plates and incubated at 37° C. overnight. Stock compound plates were prepared in advance which contained dimethyl sulfoxide (DMSO) alone and a 9-point half-log decreasing dose range of the compound in DMSO. These plates were stored at −80° C. and each plate was only thawed once and used. On day 0, the main assay plates received compound or DMSO via a sonic delivery system (ECHO). The highest final concentration of compound of the dose range in the culture plates was 10 uM. These plates were cultured at 37° C. for 3 days. A parallel set of cell line plates, which did not receive compound, were processed and read on day 0 to provide a T=0 (time zero).

On day 3, the compound treated plates were stained and fixed to measure proliferation, apoptosis, and mitotic index. A nuclear stain was used to identify cells in the wells. By counting the number of nuclei, the proliferation index of compound treated groups were calculated as a percentage relative to the DMSO control, which was set to 100%. The IC50 values were calculated using model 205 in ExcelFit. In Table 4, +=IC₅₀>100 nM; ++=IC₅₀ 50-100 nM: +++=IC₅₀<50 nM.

TABLE 4

| Cell Line | Tissue | Ex 1 |
|---|---|---|
| H4 IC₅₀ | glioma | +++ |
| SF-539 IC₅₀ | astrocytoma | +++ |
| A172 IC₅₀ | glioblastoma | +++ |
| SNB-75 IC₅₀ | glioblastoma | ++ |
| D283Med IC₅₀ | medulloblastoma | +++ |
| DBTRG-05MG IC₅₀ | glioblastoma | +++ |
| CCF-SSTG1 IC₅₀ | astrocytoma | +++ |
| SF-295 IC₅₀ | glioblastoma | +++ |
| SNB-19 IC₅₀ | glioblastoma | +++ |
| SF-268 IC₅₀ | glioblastoma | +++ |
| SW1088 IC₅₀ | astrocytoma | +++ |
| DK-MG | glioblastoma | +++ |
| U-87MG | glioblastoma; astrocytoma | +++ |

The data collected demonstrates that the compound inhibited cellular proliferation in vitro, in a number of glioma cell lines.

### IX. Washout Assay—Inhibition of Cell Proliferation and Viability by PLK1 Inhibitors

Cell growth was measured following washouts by measuring recovery from exposure to Plk1 inhibitor of the compound of Example 1. Seven glioma cell lines that can double at least once over three days were thawed and grown to 70-80% confluency before being used in the assay. On day 1, for each cell line, 2-3×10⁵ cells were seeded into each well in 6-well cell culture plates and incubated at 37° C. overnight. A stock solution and serial dilutions of the compound of Example 1 were prepared in advance which contained dimethyl sulfoxide (DMSO) alone and a 5-point dose range of the compound of Example 1 in DMSO. On day 2, the compound of Example 1 was added to each well at final concentrations of 0, 10 nM, 30 nM, 100 nM, 300 nM and 1 uM. The plates were incubated at 37° C. for 3 days.

On day 5, the compound was efficiently washed four times with culture medium supplemented with 20% fetal bovine serum (FBS) and then washed once in buffered saline solution (PBS). The cells were trypsinized. For each of the concentrations, compound-treated cell lines were plated into five 96-well plates at 5000 cells per well. For the next 4 consecutive days, one plate of the cells was taken out of the incubator and cell proliferation was measured by MTT ELISA assay.

The washout assay is designed to follow the cells' recovery from exposure to the compound. Each glioma cell line was assayed at least twice. The criteria for evaluating sensitivity or resistance was Last Growth Back Concentration (LGC), if LGC<=30 nM, the cell line is considered sensitive; if LGC>=1 uM, it is considered resistant; if LGC is in between these two concentrations, the cell line is considered intermediate. The results of the Washout Assay are reported in Table 5. Based on criteria set, all the glioma cell lines appeared to be sensitive to the compound of Example 1.

TABLE 5

| Cell Line | Tissue | LGC |
|---|---|---|
| H4 | glioma | 10 nM |
| SF-539 | astrocytoma | 30 nM |
| A172 | glioblastoma | <10 nM |
| SF-295 | glioblastoma | 10 nM |
| SNB-19 | glioblastoma | 10 nM |
| SF-268 | glioblastoma | 10 nM |
| SW1088 | astrocytoma | 10 nM |

That which is claimed is:

1. A method for the treatment of a primary CNS tumor in a mammal in need thereof comprising administering to said mammal a therapeutically effective amount of a compound of formula (I): 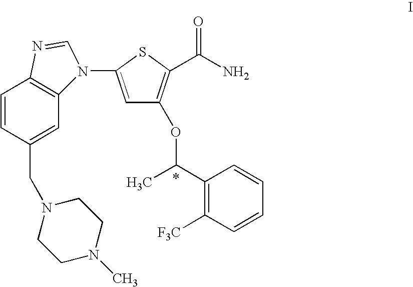
wherein * indicates a chiral carbon,
or a pharmaceutically acceptable salt thereof.

2. The method according to claim 1, wherein said compound of formula (I) is a compound of formula (I-A):

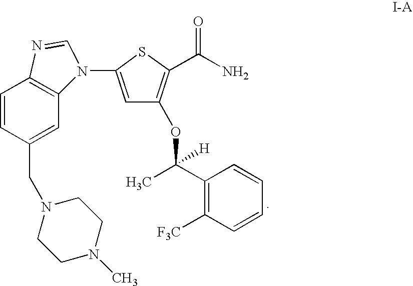

3. The method according to claims 1 wherein said primary CNS tumor is selected from tumors of neuroepithelial tissue, tumors of peripheral nerves, tumors of meninges, lymphomas and hemopoietic neoplasms, germ cell tumors, and tumors of sellar region.

4. The method according to claim 1 wherein said primary CNS tumor is a tumor of the neuroepithelial tissue.

5. A method for the treatment of an astrocytic tumor in a mammal in need thereof comprising administering to said mammal a therapeutically effective amount of a compound of formula (I): 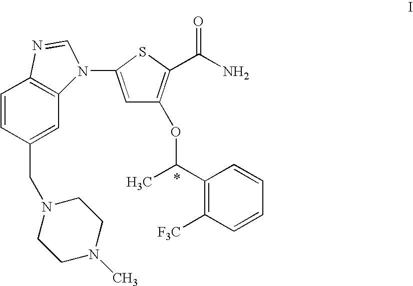
wherein * indicates a chiral carbon,
or a pharmaceutically acceptable salt thereof.

6. The method according to claim 5, wherein said compound of formula (I) is a compound of formula (I-A):

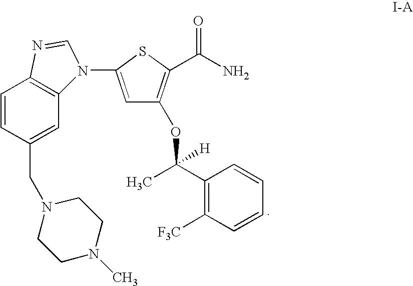

7. A method for the treatment of glioblastoma multiforme in a mammal in need thereof comprising administering to said mammal a therapeutically effective amount of a compound of formula (I): 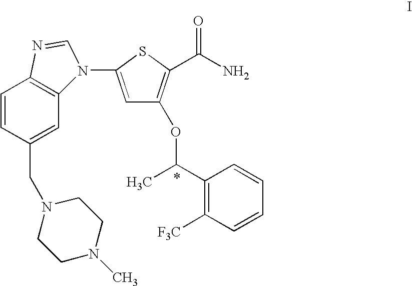
wherein * indicates a chiral carbon,
or a pharmaceutically acceptable salt thereof.

8. The method according to claim 7, wherein said compound of formula (I) is a compound of formula (I-A):

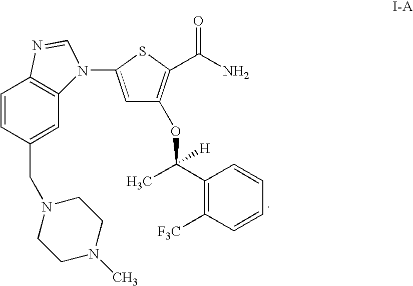

9. A method for the treatment of a metastases in the central nervous system, from a tumor originating outside of the central nervous system, in a mammal in need thereof comprising administering to said mammal a therapeutically effective amount of a compound of formula (I): 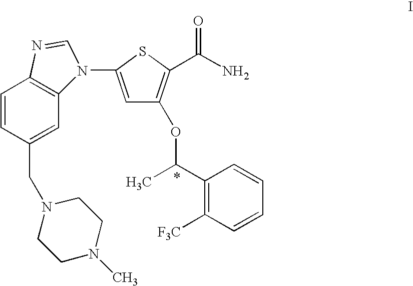
wherein * indicates a chiral carbon,
or a pharmaceutically acceptable salt thereof.

10. The method according to claim 9, wherein said compound of formula (I) is a compound of formula (I-A):

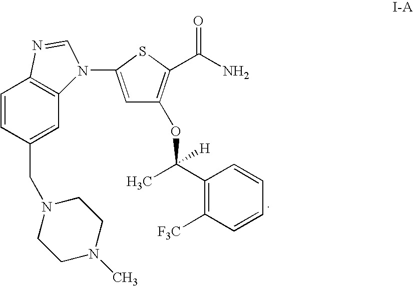

11. The method according to claim 9 wherein said metastases is selected from lung cancer metastases, breast cancer metastases, adenocarcinoma of unknown primary site metastases, melanoma metastases, renal cell cancer metastases, colon cancer metastases, sarcoma metastases, Wilm's tumor metastases, neuroblastoma metastases, germ cell tumor metastases, acute lymphoblastic leukemia metastases, high grade non-Hodgkin's lymphoma metastases and acute myeloid leukemia metastases.

12. The method according to claim 9, wherein said metastases are in tissue selected from brain, meninges, spinal cord and cerebral spinal fluid.

13. A method for treating metastases in the brain selected from lung cancer metastases, breast cancer metastases, adenocarcinoma of unknown primary site metastases, melanoma metastases, renal cell cancer metastases, colon cancer metastases, sarcoma metastases, Wilm's tumor metastases, neuroblastoma metastases, germ cell tumor metastases, acute lymphoblastic leukemia metastases, high grade non-Hodgkin's lymphoma metastases and acute myeloid leukemia metastases, in a mammal in need thereof, said method comprising administering to said mammal a therapeutically effective amount of a compound of formula (I): 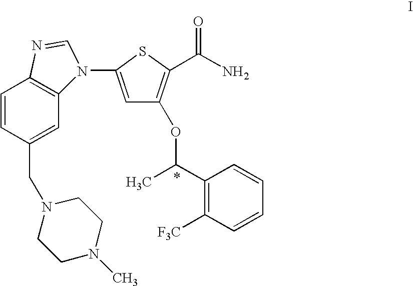
wherein * indicates a chiral carbon,
or a pharmaceutically acceptable salt thereof.

14. The method according to claim 13, wherein said compound of formula (I) is a compound of formula (I-A): [chemical structure]
